(19) United States
(12) Patent Application Publication
(10) Pub. No.: US 2026/0152761 A1
(43) Pub. Date: Jun. 4, 2026

(54) GENE THERAPY FOR TREATING WILSON'S DISEASE

(71) Applicant: The Trustees of the University of Pennsylvania, Philadelphia, PA (US)

(72) Inventors: James M. Wilson, Philadelphia, PA (US); Jenny Agnes Sidrane, Phoenixville, PA (US); Lakshmanan Govindasamy, Philadelphia, PA (US)

(21) Appl. No.: 19/351,377

(22) Filed: Oct. 7, 2025

Related U.S. Application Data
(63) Continuation of application No. 17/812,322, filed on Jul. 13, 2022, now Pat. No. 12,460,228.
Application No. 17/812,322 is a continuation of application No. 16/474,958, now Pat. No. 11,473,106.
(60) Provisional application No. 62/440,659, filed on Dec. 30, 2016.
(60) Provisional application No. 62/473,656, filed on Mar. 20, 2017.

(51) Int. Cl. C12N 15/86; A61K 48/00; C07K 14/755

(52) U.S. Cl. CPC C12N 15/86; A61K 48/00; C07K 14/755

(57) ABSTRACT

Compositions and regimens useful in treating Wilson's Disease are provided. The compositions include recombinant adeno-associated virus (rAAV) with a transthyretin enhancer and promoter driving expression of a human ATP7B.

### CROSS-REFERENCE TO RELATED APPLICATIONS

This application is a continuation of U.S. patent application Ser. No. 17/812,322, filed Jul. 13, 2022, which is continuation of U.S. patent application Ser. No. 16/474,958, filed Jun. 28, 2019, now U.S. Pat. No. 11,473,106, issued Oct. 18, 2022, which is a National Stage Entry under 35 U.S.C. 371 of International Patent Application No. PCT/US2017/068919, filed Dec. 29, 2017, which claims the benefit of priority of U.S. Provisional Patent Application No. 62/440,659, filed Dec. 30, 2016, and U.S. Provisional Patent Application No. 62/473,656 filed Mar. 20, 2017. These applications are incorporated by reference herein.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “16-7940.US.C2.xml” (created Oct. 1, 2025 and 177,785 bytes in size).

### 1. INTRODUCTION

The application relates to embodiments useful for a gene therapy for treating Wilson's Disease.

### 2. BACKGROUND

The application relates to embodiments useful for a gene therapy for treating Wilson's disease. Wilson's disease is an autosomal recessive genetic disorder copper storage disorder due to mutations in the copper-transporting ATPase 2 (ATP7B) gene on chromosome 13. Copper accumulates in tissues manifesting as neurological or psychiatric symptoms and liver disease typically observed between the ages of 12 and 23. Over time without proper treatments, high copper levels can cause life-threatening organ damage.

Current treatment approaches for Wilson's disease are daily oral therapy with chelating agents (penicillamine [Cuprimine] and trientine hydrochloride [Syprine]), zinc (to block enterocyte absorption of copper), and tetrathiomolybdate (TM), a copper chelator that forms complexes with albumin in the circulation, which require the affected individual to take medicines for their whole life. Furthermore, those treatments may cause side effects, such as drug induced lupus, myasthenia, paradoxical worsening, and do not restore normal copper metabolism. Liver transplantation is curative for Wilson's disease but transplant recipients are required to maintain a constant immune suppression regimen to prevent rejection.

### 3. SUMMARY

The embodiments described herein relate to an AAV gene therapy vector for delivering normal human copper-transporting ATPase 2 (ATP7B) to a subject in need thereof, following intravenous (IV) administration of the vector resulting in long-term, perhaps 10 years or more, of clinically meaningful correction of Wilson's Disease (“WD”). The vector dose is intended to deliver blood levels of ATP7B to reduce circulating copper levels by about 25% or more. In one embodiment, the level of circulating copper is assessed via the excretion of copper in the urine. In another embodiment, the level of circulating copper in the plasma is assessed.

In one aspect, this application provides the use of a replication deficient adeno-associated virus (AAV) to deliver a human copper-transporting ATPase 2 (ATP7B) gene to liver cells of patients (human subjects) diagnosed with WD. The recombinant AAV vector (rAAV) used for delivering the hATP7B gene (“rAAV.hATP7B”) should have a tropism for the liver (e.g., a rAAV bearing an AAV8 capsid), and the hATP7B transgene should be controlled by liver-specific expression control elements. In one embodiment, the expression control elements include one or more of the following: an enhancer; a promoter; an intron; a WPRE; and a polyA signal. Such elements are further described herein. Due to the size of the hATP7B coding sequence, selection of control elements which allow for effective expression is important. If expression of the transgene is not sufficient, the vector dosage required for correction of the defect will be too high to be practical. Thus, as described herein, selection of the e.g., enhancer, promoter and polyA is important.

In one embodiment, the hATP7B coding sequence is shown in SEQ ID NO: 1. In one embodiment, the ATP7B protein sequence is shown in SEQ ID NO: 2. The coding sequence for hATP7B is, in one embodiment, codon optimized for expression in humans. Such sequence may share less than 80% identity to the native hATP7B coding sequence (SEQ ID NO: 3). In one embodiment, the hATP7B coding sequence is that shown in SEQ ID NO: 1.

In another aspect, provided herein is an aqueous suspension suitable for administration to a WD patient which includes the rAAV described herein. In some embodiments, the suspension includes an aqueous suspending liquid and about 1×10¹² to about 1×10¹⁴ genome copies (GC) of the rAAV/mL. The suspension is, in one embodiment, suitable for intravenous injection. In other embodiment, the suspension further includes a surfactant, preservative, and/or buffer dissolved in the aqueous suspending liquid.

In another embodiment, provided herein is a method of treating a patient having WD with a rAAV as described herein. In one embodiment, about 1×10¹¹ to about 3×10¹³ genome copies (GC) of the rAAV/kg patient body weight are delivered the patient in an aqueous suspension.

### 3. BRIEF DESCRIPTION OF THE DRAWINGS

FIG. 1 is a schematic representation of AAV8.En34.TBG-S1.hATP7Bco.PA75 vector.

FIGS. 2A-2B demonstrate that urinary and serum copper levels increase over time in Atp7b KO mice. (A) Urine and (B) serum copper levels in Atp7b KO mice over time (black). Heterozygous littermates (Het) served as control (gray). Samples were collected weekly in the natural history study and inductively coupled plasma-mass spectrometry was performed to assess the copper level.

FIGS. 3A-3C demonstrate urine, copper levels, serum copper levels, and liver disease scoring in Atp7b KO mice. (A) Urine and (B) serum copper levels were evaluated by inductively coupled plasma-mass spectrometry in male and female heterozygous (Het) and Atp7b KO mice over time (KO). (C) Atp7b KO mice were necropsied at 2, 3, 4, 5, 9, 10, and 12 months of age. Liver was harvested, stained with H&E, and evaluated histologically according to the 1-5 scoring system. Values expressed as mean±SEM.

FIG. 4 demonstrates Timm's staining in Atp7b KO mice. Representative result of Timm's staining for copper in the liver of a 2 month-old Atp7b KO mouse with identification number 1345. Black deposits indicate positive staining for copper. A 3.7 month-old wild type mouse with identification number 307 served as a negative control.

FIG. 5 demonstrates that liver disease develops over time in Atp7b KO mice. Atp7b KO mice were necropsied at 2, 3, 4, 5, 9, 10, and 12 months of age and liver was harvested to evaluate liver disease. H&E, Sirius Red, and Timm's stain were performed for histopathologic evaluation of liver lesions, including fibrosis, and copper accumulation. *, area of regeneration within the liver of a 12 month old Atp7b KO mouse. WT, section from a wild type mouse as a negative control for copper accumulation as seen by the Timm's stain.

FIGS. 6A-6C demonstrate serum chemistry values in 8 month old Atp7b KO mice. (A) ALT, (B) AST, and (C) total bilirubin levels in 8 month old Atp7b KO mice (KO) and heterozygous mice (Het).

FIG. 7 demonstrates liver disease in 8 month old Atp7b KO mice. Atp7b KO and heterozygous (Het) mice were necropsied at 8 months of age. Liver was harvested, stained with H&E, and evaluated histologically according to the 1-5 scoring system. Values expressed as mean±SEM.

FIGS. 8A and 8B demonstrate liver fibrosis and copper accumulation in 8 month old Atp7b KO mice. Atp7b KO (KO) and heterozygous (Het) mice were necropsied at 8 months of age. Liver was harvested, stained with Sirius Red and Timm's stain for evaluation of fibrosis and copper accumulation, respectively. Histopathologic evaluation of liver lesions, including (A) fibrosis and (B) copper accumulation, was performed according to the 1-5 scoring system. Values expressed as mean±SEM.

FIG. 9 demonstrates liver copper levels in 8 month old Atp7b KO mice Atp7b KO (KO) and heterozygous (Het) mice were necropsied at 8 months of age. Liver was harvested and liver copper levels were evaluated by inductively coupled plasma-mass spectrometry. Values expressed as mean±SEM.

FIGS. 10A-10C demonstrates that AAV8 gene therapy can restore normal liver copper metabolism in Atp7b KO mice. Male Atp7b KO mice were injected i.v. with 10¹¹ GC/mouse and 10¹⁰ GC/mouse of AAV8.TTR.hATP7Bco, and female Atp7b KO mice were injected i.v. with 10¹¹, 10¹⁰, and 10⁹ GC/mouse of the same vector. Serum copper levels in (A) males and (B) females were evaluated by inductively coupled plasma-mass spectrometry and compared to serum copper levels from age-matched male and female heterozygous (het) and Atp7b KO mice. Mice were necropsied at 9 months of age and liver was harvested. (C) Liver copper levels were also evaluated by inductively coupled plasma-mass spectrometry and compared to age-matched uninjected heterozygous (het) and Atp7b KO mice. Values expressed as mean±SEM. ns, not significant; **p<0.01, ****p<0.0001.

FIG. 11 demonstrates that high-dose AAV8 gene therapy prevents the development of liver disease in Atp7b KO mice. Male Atp7b KO mice were injected i.v. with 10¹¹ GC/mouse and 10¹⁰ GC/mouse of AAV8.TTR.hATP7Bco, and female Atp7b KO mice were injected i.v. with 10¹¹, 10¹⁰, and 10⁹ GC/mouse of the same vector. Mice were necropsied at 9 months of age and liver was harvested to evaluate liver disease. H&E, Sirius Red, and Timm's stain were performed to evaluate histopathological lesions of the liver, including, fibrosis and copper accumulation. Images from age-matched uninjected Atp7b KO mice are included for comparison (also presented in FIG. 2). WT, section from a wild type (WT) mouse as a negative control for copper accumulation as seen by the Timm's stain. KO, section from a two month old Atp7b KO mouse as a positive control for copper accumulation as seen by the Timm's stain.

FIGS. 12A-12C demonstrates quantification of prevention of liver disease following high-dose AAV8 gene therapy in Atp7b KO mice. Male Atp7b KO mice were injected i.v. with 10¹¹ GC/mouse and 10¹⁰ GC/mouse of AAV8.TTR.hATP7Bco and female Atp7b KO mice were injected i.v. with 10¹¹, 10¹⁰, and 10⁹ GC/mouse of the same vector. Mice were necropsied at 9 months of age and liver was harvested for histologic to evaluation. (A) Liver sections were stained with H&E and evaluated histologically according to the 1-5 scoring system. (B) Liver sections were stained with Sirius Red and evaluated for fibrosis according to the 1-3 scoring system and (C) Timm's stain was performed on liver sections for evaluation of copper accumulation according to the 1-5 scoring system. Values expressed as mean±SEM were compared to age-matched uninjected heterozygous (Het) and Atp7b KO mice. ns, not significant; *p<0.05, **p<0.01, ***p<0.001, ****p<0.0001.

FIGS. 13A-13C demonstrate serum chemistry levels in Atp7b KO mice following AAV8 gene therapy. Male Atp7b KO mice were injected IV with 10¹¹ GC/mouse and 10¹⁰ GC/mouse of AAV8.TTR.hATP7Bco and female Atp7b KO mice were injected i.v. with 10¹¹, 10¹⁰, and 10⁹ GC/mouse of the same vector. (A) ALT, (B) AST, and (C) total bilirubin levels were evaluated in serum.

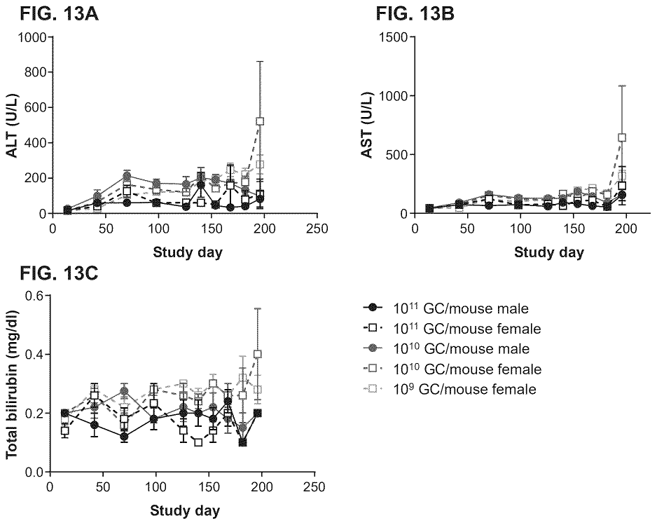

FIG. 14 demonstrates detection of ceruloplasmin by Western blot. Western blot detecting copper-bound (Holo, lower band) and non-copper-bound (Apo, upper band) forms of ceruloplasmin in Atp7b KO mice injected i.v. with 10¹⁰ or 10¹¹ GC per mouse of AAV8.EnTTR.TTR.hATP7Bco.PA75. Blood samples were collected on Day 21 after the administration. Protein markers were provided in the center lane for comparison. Atp7b KO and heterozygous (het) littermates without vector injections served as controls (1318, 6 month old het; 1313, 6 month old Atp7b KO; 388, Atp7b KO).

FIGS. 15A-15B demonstrates serum copper levels in female and male AAV8 vector administered Atp7b KO mice. Atp7b KO mice were administered i.v. with 3×10¹² GC/kg of AAV8 vectors for expression of hATP7Bco with different enhancer/promoter combinations (circles, EnTTR.TTR, AAV8.EnTTR.TTR.hATP7Bco.PA75; squares, En34.TBG-S1, AAV8.En34.TBG-S1.hATP7Bco.PA75; upright triangles, En34.TTR, AAV8.En34.TTR.hATP7Bco.PA75; inverted triangles, ABPS.TBG-S1, AAV8.ABPS.TBG-S1.hATP7Bco.PA75; diamonds, En34.mTTR, AAV8.En34.mTTR.hATP7Bco.PA75). Uninjected Atp7b KO and heterozygous (Het) mice served as controls. Serum copper levels were evaluated by inductively coupled plasma-mass spectrometry in (A) female and (B) male Atp7b KO mice. Values expressed as mean±SEM.

FIGS. 16A-16C demonstrates serum chemistries in female and male AAV8 vector administered Atp7b KO mice. Female and male Atp7b KO mice were administered i.v. with 3×10¹² GC/kg of AAV8 vectors for expression of hATP7Bco with different enhancer/promoter combinations (EnTTR.TTR, AAV8.EnTTR.TTR.hATP7Bco.PA75; En34.TBG-S1, AAV8.En34.TBG-S1.hATP7Bco.PA75; En34.TTR, AAV8.En34.TTR.hATP7Bco.PA75; ABPS.TBG-S1, AAV8.ABPS.TBG-S1.hATP7Bco.PA75; En34.mTTR, AAV8.En34.mTTR.hATP7Bco.PA75). Vehicle control administered Atp7b KO (PBS) mice served as controls. (A) ALT, (B) AST, and (C) total bilirubin levels were evaluated in serum. Values expressed as mean±SEM.

FIG. 17 demonstrates liver ATP7Bco expression determined by Western blot. Female and male Atp7b KO mice were administered i.v. with 3×10¹² GC/kg of AAV8 vectors for expression of hATP7Bco with different enhancer/promoter combinations (EnTTR.TTR, AAV8.EnTTR.TTR.hATP7Bco.PA75; En34.TBG-S1, AAV8.En34.TBG-S1.hATP7Bco.PA75; En34.TTR, AAV8.En34.TTR.hATP7Bco.PA75; ABPS.TBG-S1, AAV8.ABPS.TBG-S1.hATP7Bco.PA75; En34.mTTR, AAV8.En34.mTTR.hATP7Bco.PA75) and sacrificed at 6 months of age. Western blot detecting ATP7B in Atp7b KO mice was quantified by band densitometry. Values expressed as mean±SEM.

FIG. 18 demonstrates liver disease in female and male AAV8 vector administered Atp7b KO mice. Female and male Atp7b KO mice were administered i.v. with 3×10¹² GC/kg of AAV8 vectors for expression of hATP7Bco with different enhancer/promoter combinations (EnTTR.TTR, AAV8.EnTTR.TTR.hATP7Bco.PA75; En34.TBG-S1, AAV8.En34.TBG-S1.hATP7Bco.PA75; En34.TTR, AAV8.En34.TTR.hATP7Bco.PA75; ABPS.TBG-S1, AAV8.ABPS.TBG-S1.hATP7Bco.PA75; En34.mTTR, AAV8.En34.mTTR.hATP7Bco.PA75) and sacrificed at 6 months of age. Vehicle control administered Atp7b KO (PBS) mice served as controls. Liver sections were stained with H&E and evaluated histologically according to the 1-5 scoring system. Values expressed as mean±SEM.

FIGS. 19A-19B demonstrate liver fibrosis and copper accumulation in female and male AAV8 vector administered Atp7b KO mice. Female and male Atp7b KO mice were administered i.v. with 3×10¹² GC/kg of AAV8 vectors for expression of hATP7Bco with different enhancer/promoter combinations (EnTTR.TTR, AAV8.EnTTR.TTR.hATP7Bco.PA75; En34.TBG-S1, AAV8.En34.TBG-S1.hATP7Bco.PA75; En34.TTR, AAV8.En34.TTR.hATP7Bco.PA75; ABPS.TBG-S1, AAV8.ABPS.TBG-S1.hATP7Bco.PA75; En34.mTTR, AAV8.En34.mTTR.hATP7Bco.PA75) and sacrificed at 6 months of age. Vehicle control administered Atp7b KO (PBS) mice served as controls. (A) Liver sections were stained with Sirius Red and evaluated for fibrosis according to the 1-3 scoring system and (B) Timm's stain was performed on liver sections for evaluation of copper accumulation according to the 1-5 scoring system. Values expressed as mean±SEM were compared to age-matched uninjected heterozygous (Het) and Atp7b KO (KO) mice.

FIG. 20 demonstrates liver copper levels in female and male AAV8 vector administered Atp7b KO mice. Female and male Atp7b KO mice were administered i.v. with 3×10¹² GC/kg of AAV8 vectors for expression of hATP7Bco with different enhancer/promoter combinations (EnTTR.TTR, AAV8.EnTTR.TTR.hATP7Bco.PA75; En34.TBG-S1, AAV8.En34.TBG-S1.hATP7Bco.PA75; En34.TTR, AAV8.En34.TTR.hATP7Bco.PA75; ABPS.TBG-S1, AAV8.ABPS.TBG-S1.hATP7Bco.PA75; En34.mTTR, AAV8.En34.mTTR.hATP7Bco.PA75) and sacrificed at 6 months of age. Vehicle control administered Atp7b KO (PBS) mice served as controls. Liver copper levels were evaluated by inductively coupled plasma-mass spectrometry and compared to age-matched uninjected heterozygous (het) and Atp7b KO (KO) mice. Values expressed as mean±SEM.

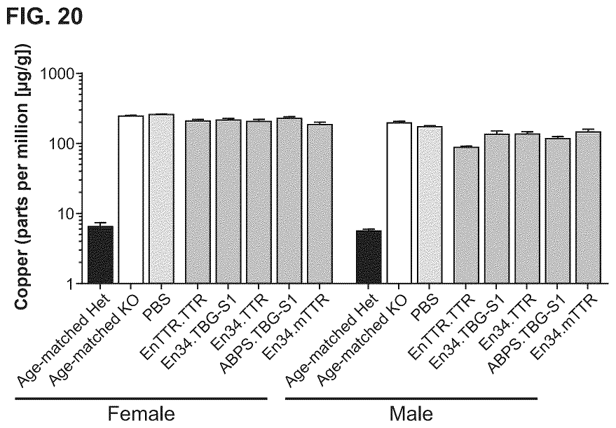

FIG. 21 demonstrates detection of ceruloplasmin by Western blot in male AAV8 vector administered Atp7b KO mice. Western blot detecting copper-bound (Holo, lower band) and non-copper-bound (Apo, upper band) forms of ceruloplasmin in male Atp7b KO mice were administered i.v. with 3×10¹² GC/kg of AAV8 vectors for expression of hATP7Bco with different enhancer/promoter combinations (1, AAV8.EnTTR.TTR.hATP7Bco.PA75; 2, AAV8.En34.TBG-S1.hATP7Bco.PA75; 3, AAV8.En34.TTR.hATP7Bco.PA75; 4, AAV8.ABPS.TBG-S1.hATP7Bco.PA75). Blood samples were collected on Day 21 after the administration. Protein markers were provided in the center lane for comparison. Atp7b KO, heterozygous (het), and wild type (WT) littermates without vector injections served as controls (1313, 6 month old Atp7b KO; 1318, 6 month old het; 1345, 2 month old Atp7b KO; 1945, WT).

FIG. 22 demonstrates serum copper levels in male AAV8 truncated ATP7B vector administered Atp7b KO mice. Male Atp7b KO mice were administered i.v. with 3×10¹² GC/kg of AAV8 vectors for expression of different truncated versions of hATP7Bco (MBDIDel, AAV8.En34.TBG-S1.hATP7BcoMBDIDel.PA75; MBD2Del, AAV8.En34.TBG-S1.hATP7BcoMBD2Del.PA75; MBD3Del, AAV8.En34.TBG-S1.hATP7BcoMBD3Del.PA75; MBD1-2Del, AAV8.En34.TBG-S1.hATP7BcoMBD1-2Del.PA75; MBD1-4Del, AAV8.En34.TBG-S1.hATP7BcoMBD1-4Del.PA75; MBD1-5Del, AAV8.En34.TBG-S1.hATP7BcoMBD1-5Del.PA75; TBG-MBD1-4Del, AAV8.TBG.hATP7BcoMBD1-4Del.PA75; TBG-MBD1-5Del, AAV8.TBG.hATP7BcoMBD1-5Del.PA75). Serum copper levels were evaluated by inductively coupled plasma-mass spectrometry and compared to heterozygous (Het) and Atp7b KO mice (KO) over time. Values expressed as mean±SEM.

FIGS. 23A-23C demonstrates serum chemistries in male AAV8 truncated ATP7B vector administered Atp7b KO mice. Male Atp7b KO mice were administered i.v. with 3×10¹² GC/kg of AAV8 vectors for expression of different truncated versions of hATP7Bco (MBDIDel, AAV8.En34.TBG-S1.hATP7BcoMBDIDel.PA75; MBD2Del, AAV8.En34.TBG-S1.hATP7BcoMBD2Del.PA75; MBD3Del, AAV8.En34.TBG-S1.hATP7BcoMBD3Del.PA75; MBD1-2Del, AAV8.En34.TBG-S1.hATP7BcoMBD1-2Del.PA75; MBD1-4Del, AAV8.En34.TBG-S1.hATP7BcoMBD1-4Del.PA75; MBD1-5Del, AAV8.En34.TBG-S1.hATP7BcoMBD1-5Del.PA75; TBG-MBD1-4Del, AAV8.TBG.hATP7BcoMBD1-4Del.PA75; TBG-MBD1-5Del, AAV8.TBG.hATP7BcoMBD1-5Del.PA75). Mice were necropsied at 6 months of age and (A) ALT, (B) AST, and (C) total bilirubin levels were evaluated in serum. Values expressed as mean±SEM.

FIG. 24 demonstrates liver ATP7Bco expression determined by Western blot in male AAV8 truncated ATP7B vector administered Atp7b KO mice. Male Atp7b KO mice were administered i.v. with 3×10¹² GC/kg of AAV8 vectors for expression of different truncated versions of hATP7Bco (MBDIDel, AAV8.En34.TBG-S1.hATP7BcoMBDIDel.PA75; MBD2Del, AAV8.En34.TBG-S1.hATP7BcoMBD2Del.PA75; MBD3Del, AAV8.En34.TBG-S1.hATP7BcoMBD3Del.PA75; MBD1-2Del, AAV8.En34.TBG-S1.hATP7BcoMBD1-2Del.PA75; MBD1-4Del, AAV8.En34.TBG-S1.hATP7BcoMBD1-4Del.PA75; MBD1-5Del, AAV8.En34.TBG-S1.hATP7BcoMBD1-5Del.PA75; TBG-MBD1-4Del, AAV8.TBG.hATP7BcoMBD1-4Del.PA75; TBG-MBD1-5Del, AAV8.TBG.hATP7BcoMBD1-5Del.PA75). Mice were necropsied at 6 months of age. Western blot detecting ATP7B in Atp7b KO mice was quantified by band densitometry. Values expressed as mean±SEM.

FIG. 25 demonstrates liver disease in male AAV8 truncated ATP7B vector administered Atp7b KO mice. Male Atp7b KO mice were administered i.v. with 3×10¹² GC/kg of AAV8 vectors for expression of different truncated versions of hATP7Bco (MBDIDel, AAV8.En34.TBG-S1.hATP7BcoMBD1Del.PA75; MBD2Del, AAV8.En34.TBG-S1.hATP7BcoMBD2Del.PA75; MBD3Del, AAV8.En34.TBG-S1.hATP7BcoMBD3Del.PA75; MBD1-2Del, AAV8.En34.TBG-S1.hATP7BcoMBD1-2Del.PA75; MBD1-4Del, AAV8.En34.TBG-S1.hATP7BcoMBD1-4Del.PA75; MBD1-5Del, AAV8.En34.TBG-S1.hATP7BcoMBD1-5Del.PA75; TBG-MBD1-4Del, AAV8.TBG.hATP7BcoMBD1-4Del.PA75; TBG-MBD1-5Del, AAV8.TBG.hATP7BcoMBD1-5Del.PA75). Mice were necropsied at 6 months of age. Vehicle control administered Atp7b KO (PBS) mice served as controls. Liver sections were stained with H&E and evaluated histologically according to the 1-5 scoring system. Values expressed as mean±SEM.

FIGS. 26A-26B demonstrate liver fibrosis and copper accumulation in male AAV8 truncated ATP7B vector administered Atp7b KO mice. Male Atp7b KO mice were administered i.v. with 3×10¹² GC/kg of AAV8 vectors for expression of different truncated versions of hATP7Bco (MBDIDel, AAV8.En34.TBG-S1.hATP7BcoMBDIDel.PA75; MBD2Del, AAV8.En34.TBG-S1.hATP7BcoMBD2Del.PA75; MBD3Del, AAV8.En34.TBG-S1.hATP7BcoMBD3Del.PA75; MBD1-2Del, AAV8.En34.TBG-S1.hATP7BcoMBD1-2Del.PA75; MBD1-4Del, AAV8.En34.TBG-S1.hATP7BcoMBD1-4Del.PA75; MBD1-5Del, AAV8.En34.TBG-S1.hATP7BcoMBD1-5Del.PA75; TBG-MBD1-4Del, AAV8.TBG.hATP7BcoMBD1-4Del.PA75; TBG-MBD1-5Del, AAV8.TBG.hATP7BcoMBD1-5Del.PA75). Mice were necropsied at 6 months of age. Vehicle control administered Atp7b KO (PBS) mice served as controls. (A) Liver sections were stained with Sirius Red and evaluated for fibrosis according to the 1-3 scoring system and (B) Timm's stain was performed on liver sections for evaluation of copper accumulation according to the 1-5 scoring system. Values expressed as mean±SEM were compared to age-matched uninjected heterozygous (Het) and Atp7b KO (KO) mice.

FIG. 27 demonstrates liver copper levels in male AAV8 truncated ATP7B vector administered Atp7b KO mice. Male Atp7b KO mice were administered i.v. with 3×10¹² GC/kg of AAV8 vectors for expression of different truncated versions of hATP7Bco (MBDIDel, AAV8.En34.TBG-S1.hATP7BcoMBDIDel.PA75; MBD2Del, AAV8.En34.TBG-S1.hATP7BcoMBD2Del.PA75; MBD3Del, AAV8.En34.TBG-S1.hATP7BcoMBD3Del.PA75; MBD1-2Del, AAV8.En34.TBG-S1.hATP7BcoMBD1-2Del.PA75; MBD1-4Del, AAV8.En34.TBG-S1.hATP7BcoMBD1-4Del.PA75; MBD1-5Del, AAV8.En34.TBG-S1.hATP7BcoMBD1-5Del.PA75; TBG-MBD1-4Del, AAV8.TBG.hATP7BcoMBD1-4Del.PA75; TBG-MBD1-5Del, AAV8.TBG.hATP7BcoMBD1-5Del.PA75). Mice were necropsied at 6 months of age. Vehicle control administered Atp7b KO (PBS) mice served as controls. Liver copper levels were evaluated by inductively coupled plasma-mass spectrometry and compared to age-matched uninjected heterozygous (het) and Atp7b KO (KO) mice. Values expressed as mean±SEM were compared to age-matched uninjected heterozygous (Het) and Atp7b KO (KO) mice.

### 4. DETAILED DESCRIPTION

The embodiments described in the application relate to the use of a replication deficient adeno-associated virus (AAV) to deliver a human copper-transporting ATPase 2 (ATP7B) gene to liver cells of patients (human subjects) diagnosed with Wilson's Disease (WD). The recombinant AAV vector (rAAV) used for delivering the hATP7B gene (“rAAV.hATP7B”) should have a tropism for the liver (e.g., an rAAV bearing an AAV8 capsid), and the hATP7B transgene should be controlled by liver-specific expression control elements. In one embodiment, the expression control elements include one or more of the following: an enhancer; a promoter; an intron; a WPRE; and a polyA signal. Such elements are further described herein.

As used herein, “AAV8 capsid” refers to the AAV8 capsid having the amino acid sequence of GenBank, accession: YP_077180.1, SEQ ID NO: 16, which is incorporated by reference herein. Some variation from this encoded sequence is permitted, which may include sequences having about 99% identity to the referenced amino acid sequence in YP_077180.1 and WO 2003/052051 (which is incorporated herein by reference) (i.e., less than about 1% variation from the referenced sequence). Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2015/0315612.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The terms “percent (%) identity”, “sequence identity”, “percent sequence identity”, or “percent identical” in the context of amino acid sequences refers to the residues in the two sequences which are the same when aligned for correspondence. Percent identity may be readily determined for amino acid sequences over the full-length of a protein, polypeptide, about 32 amino acids, about 330 amino acids, or a peptide fragment thereof or the corresponding nucleic acid sequence coding sequencers. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein, the term “operably linked” refers to both expression control sequences that are contiguous with the gene of interest and expression control sequences that act in trans or at a distance to control the gene of interest.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### 5.1 Gene Therapy Vectors

In one aspect, a recombinant adeno-associated virus (rAAV) vector carrying the human ATP7B gene is provided for use in gene therapy. The rAAV.hATP7B vector should have a tropism for the liver (e.g., a rAAV bearing an AAV8 capsid) and the hATP7B transgene should be controlled by liver-specific expression control elements. The vector is formulated in a buffer/carrier suitable for infusion in human subjects. The buffer/carrier should include a component that prevents the rAAV from sticking to the infusion tubing but does not interfere with the rAAV binding activity in vivo.

### 5.1.1. The rAAV.hATP7B Vector

### 5.1.1.1. The hATP7B Sequence

Wilson's Disease is an inherited error of metabolism caused predominantly by mutations in the ATP7B gene, which encodes a copper-transporting P-type ATPase. ATP7B is responsible for transporting copper from intracellular chaperone proteins into the secretory pathway, both for excretion into bile and for incorporation into apo-ceruloplasmin for the synthesis of functional ceruloplasmin. The development of Wilson's disease is due to the accumulation of copper in affected tissues. See, EASL Clinical Practice Guidelines: Wilson's disease, EASL Journal of Hepatology, 2012, 56(671-85), which is incorporated herein by reference.

The clinical hallmark of Wilson's disease is the Kayser-Fleischer ring, which is present in 95% of patients with neurologic symptoms and somewhat over half of those without neurologic symptoms. Neurologic signs are variable, most often tremor, ataxia, and dystonia. Any type of liver disease may be encountered in patients with Wilson's disease. Clinically evident liver disease may precede neurologic manifestations by as much as 10 years and most patients with neurologic symptoms have some degree of liver disease at presentation. Presenting symptoms of liver disease can be highly variable, ranging from asymptomatic, with only biochemical abnormalities, to overt cirrhosis with all its complications. Wilson's disease may also present as acute hepatic failure sometimes associated with Coombs-negative hemolytic anemia and acute renal failure. The following table 1 provides a prognostic index in WD. (See, EASL Clinical Practice Guidelines: Wilson's disease, EASL Journal of Hepatology, 2012, 56(671-85), which is incorporated herein by reference.)

| | 1* | 2* | 3* | 4* |
|---|---|---|---|---|
| Serum bilirubin | 100-150 | 151-200 | 201-300 | >300 |
| (μmol/L) | | | | |
| AST (U/L) | 100-150 | 151-300 | 301-400 | >400 |
| INR | 1.3-1.6 | 1.7-1.9 | 2.0-2.4 | >2.4 |
| WBC [10⁹/L] | 6.8-8.3 | 8.4-10.3 | 10.4-15.3 | >15.3 |
| Albumin [g/L] | 34-44 | 25-33 | 21-24 | <21 |

*= score points, upper limit of normal for AST = 20 IU/ml (at King’s College). A score ≥11 is associated with high probability of death without liver transplantation.

ATP7B has eight transmembrane domains that form a path through cell membranes for copper translocation; and a large N-terminus with six metal-binding domains (MBDs), each comprising approximately 70 amino acids and the highly conserved metal-binding motif GMxCxxC (where x is any amino acid). Other domains include the intramembrane CPC motif that is required for copper translocation through the membrane, the N-domain containing the ATP-binding site, the P-domain containing the conserved aspartic acid residue and the A-domain comprising the phosphatase domain. Various mutations in the hATP7B gene and/or resulting protein are known which are present in some or all patients with Wilson's Disease. A complete listing of the known mutations contributing to WB can be found at online at uniprot.org/uniprot/P35670, which is incorporated herein by reference. Further, in addition to the canonical sequence (also called isoform a, which is the longest isoform; NCBI Reference Sequence: NP_000044.2), four additional isoforms are known: NCBI Reference Sequences NP_001005918.1, NP_001230111.1, NP_001317507.1, NP_001317508.1, each of which is incorporated herein by reference. The compositions and methods described herein may be used to treat subjects having any ATP7B variant protein which causes disease.

In one embodiment, the hATP7B gene encodes the hATP7B protein shown in SEQ ID NO: 2. Thus, in one embodiment, the hATP7B transgene can include, but is not limited to, the sequence provided by SEQ ID NO: 1 or SEQ ID NO: 3 which are provided in the attached Sequence Listing, which is incorporated by reference herein. SEQ ID NO: 3 provides the cDNA for native human ATP7B. SEQ ID NO: 1 provides an engineered cDNA for human ATP7B, which has been codon optimized for expression in humans (sometimes referred to herein as hATP7Bco). It is to be understood that reference to hATP7B herein may, in some embodiments, refer to the hATP7B native or codon optimized sequence, or any of the variants described herein. Alternatively or additionally, web-based or commercially available computer programs, as well as service based companies may be used to back translate the amino acid sequences to nucleic acid coding sequences, including both RNA and/or cDNA. See, e.g., backtranseq by EMBOSS, ebi.ac.uk/Tools/st/; Gene Infinity (geneinfinity.org/sms-/sms_backtranslation.html); ExPasy (expasy.org/tools/). It is intended that all nucleic acids encoding the described hATP7B polypeptide sequences are encompassed, including nucleic acid sequences which have been optimized for expression in the desired target subject (e.g., by codon optimization).

The native coding sequence of ATP7B is over 4.3 kb (SEQ ID NO: 3; Genbank Accession number XM_005266430), resulting in a 1465 amino acid protein (SEQ ID NO: 2). Due to the large size of ATP7B, and the packaging capacity of viral vectors, including the AAV vector, in some embodiments, it is desirable that the ATP7B coding sequence is shortened. It has been shown that deletion of the first 5 MBD showed a level of catalytic phosphorylation of the resulting protein consistent with wild type. See, Huster and Lutsenko, J. Biological Chem, June 2003, which is incorporated herein by reference. Thus, in one embodiment, the ATP7B coding sequence is shortened by deleting one or more MDB. In one embodiment, the ATP7B coding sequence has MBD1-2 deleted (e.g., as shown in SEQ ID NO: 17 and nt 403 to nt 4368 of SEQ ID NO: 35). In another embodiment, the ATP7B coding sequence has MBD1-3 deleted. In another embodiment, the ATP7B coding sequence has MBD1-4 deleted (e.g., as shown in SEQ ID NO: 18, and nt 403 to nt 3762 of SEQ ID NO: 34 and nt 1059 to nt 4418 of SEQ ID NO: 29). In another embodiment, the ATP7B coding sequence has MBD1-5 deleted (e.g., as shown in SEQ ID NO: 19, nt 403 to nt 3369 of SEQ ID NO: 33 and nt 1059 to nt 4025 of SEQ ID NO: 28). In another embodiment, the ATP7B coding sequence has MBD1 deleted (e.g., as shown in SEQ ID NO: 20 and nt 403 to nt 4686 of SEQ ID NO: 32). In another embodiment, the ATP7B coding sequence has MBD2 deleted (e.g., as shown in SEQ ID NO: 21 and nt 403 to nt 4617 of SEQ ID NO: 31). In another embodiment, the ATP7B coding sequence has MBD3 deleted (e.g., as shown in SEQ ID NO: 22 and nt 403 to nt 4719 of SEQ ID NO: 30). In another embodiment, the ATP7B coding sequence has MBD1-4 and 6 deleted (e.g., as described by Cater et al, Biochem J. 2004 Jun. 15; 380(Pt 3): 805-813, which is incorporated herein by reference). See also, Gourdon et al, Biol Chem. 2012 April; 393(4):205-16; Lutsenko, S., et al. (2007). “Function and regulation of human copper-transporting ATPases.” Physiological reviews 87(3): 1011-1046; Safaci, R., et al. (2013). “The role of metal binding and phosphorylation domains in the regulation of cisplatin-induced trafficking of ATP7B.” Metallomics 5(8): 964-972; and US Patent Publication No. 2015/0045284, each of which is incorporated herein by reference.

In one embodiment, the nucleic acid sequence encoding hATP7B shares at least 95% identity with the native hATP7B coding sequence of SEQ ID NO: 3 or SEQ ID NO: 1, or any of the variants shown in SEQ ID NO: 17, SEQ ID NO: 18, SEQ ID NO: 19, SEQ ID NO: 20, SEQ ID NO: 21, or SEQ ID NO: 22. In another embodiment, the nucleic acid sequence encoding hATP7B shares at least 90, 85, 80, 75, 70, or 65% identity with the native hATP7B coding sequence of SEQ ID NO: 3 or SEQ ID NO: 1, or any of the variants shown in SEQ ID NO: 17, SEQ ID NO: 18, SEQ ID NO: 19, SEQ ID NO: 20, SEQ ID NO: 21, or SEQ ID NO: 22. In one embodiment, the nucleic acid sequence encoding hATP7B shares about 79% identity with the native hATP7B coding sequence of SEQ ID NO: 3 or SEQ ID NO: 1, or any of the variants shown in SEQ ID NO: 17, SEQ ID NO: 18, SEQ ID NO: 19, SEQ ID NO: 20, SEQ ID NO: 21, or SEQ ID NO: 22. In one embodiment, the nucleic acid sequence encoding hATP7B is SEQ ID NO: 1. In another embodiment, the nucleic acid sequence encoding hATP7B is SEQ ID NO: 17. In another embodiment, the nucleic acid sequence encoding hATP7B is SEQ ID NO: 18. In another embodiment, the nucleic acid sequence encoding hATP7B is SEQ ID NO: 19. In another embodiment, the nucleic acid sequence encoding hATP7B is SEQ ID NO: 20. In another embodiment, the nucleic acid sequence encoding hATP7B is SEQ ID NO: 21. In another embodiment, the nucleic acid sequence encoding hATP7B is SEQ ID NO: 22.

Codon-optimized coding regions can be designed by various different methods. This optimization may be performed using methods which are available on-line (e.g., GeneArt,), published methods, or a company which provides codon optimizing services, e.g., as DNA2.0 (Menlo Park, CA). One codon optimizing approach is described, e.g., in International Patent Publication No. WO 2015/012924, which is incorporated by reference herein. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Thermo Fisher Scientific Inc. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

The goal of therapies described herein would provide functional ATP7B enzyme resulting in a reduction of serum copper levels of 25% or more. In one embodiment, urinary copper excretion of 3-8 μmol or less per 24 hours is desirable.

Primary/secondary goals of the therapies described herein include, without limitation:
Normalization of serum non-ceruloplasmin bound copper (NCC) (<150microg/L)
Normalization of serum aminotransferase (liver biochemistries, ALT/AST)
Normalization of urinary Cu (<40 microg/24 hours (0.6 micromol/24 hours) ULN)
Normalization of serum ceruloplasmin (>200 mg/L) [can be inconsistent]
Improvement of Clinician Global Impression (CGI) scale [1: severity & 2: global improvement)
Incidence of AEs

### Exploratory:

⁶⁵Cu, a nonradioactive isotope for copper which can be detected by mass spec
Improvement in IQ, neurocognitive and psychiatric functions (Unified Wilson's Disease Rating Scale (UWDRS) & Mini International Neuropsychiatric Interview (M.I.N.I.)); and
PROs (EQ5D, MMAS-8, TSQM)

In one embodiment, the “subject” or “patient” is a mammalian subject having WD as described above. It is intended that a patient having WD of any severity is the intended subject.

### 5.1.1.2. The rAAV Vector

Because ATP7B is natively expressed in the liver, it is desirable to use an AAV which shows tropism for liver. In one embodiment, the AAV supplying the capsid is AAV8. In another embodiment, the AAV supplying the capsid is AAVrh.10. In yet another embodiment, the AAV supplying the capsid is a Clade E AAV. Such AAV include rh.2; rh. 10; rh. 25; bb.1, bb.2, pi.1, pi.2, pi.3, rh.38, rh.40, rh.43, rh.49, rh.50, rh.51, rh.52, rh.53, rh.57, rh.58, rh.61, rh.64, hu.6, hu. 17, hu.37, hu.39, hu.40, hu.41, hu.42, hu.66, and hu.67. This clade further includes modified rh. 2; modified rh. 58; and modified rh.64. See, WO 2005/033321, which is incorporated herein by reference. However, any of a number of rAAV vectors with liver tropism can be used.

In a specific embodiment described in the Examples, infra, the gene therapy vector is an AAV8 vector expressing an hATP7B transgene under control of a thyroxine binding globulin (TBG-S1) promoter referred to as AAV8.En34.TBG-S1.hATP7Bco.PA75. The external AAV vector component is a serotype 8, T=1 icosahedral capsid consisting of 60 copies of three AAV viral proteins, VP1, VP2, and VP3, at a ratio of 1:1:10. The capsid contains a single-stranded DNA rAAV vector genome.

In one embodiment, the rAAV.hATP7B genome contains an hATP7B transgene flanked by two AAV inverted terminal repeats (ITRs). In one embodiment, the hATP7B transgene includes one or more of an enhancer, promoter, an hATP7B coding sequence, and polyadenylation (polyA) signal. These control sequences are “operably linked” to the hATP7B gene sequences. The expression cassette containing these sequences may be engineered onto a plasmid which is used for production of a viral vector.

The ITRs are the genetic elements responsible for the replication and packaging of the genome during vector production and are the only viral cis elements required to generate rAAV. The minimal sequences required to package the expression cassette into an AAV viral particle are the AAV 5′ and 3′ ITRs, which may be of the same AAV origin as the capsid, or which of a different AAV origin (to produce an AAV pseudotype). In one embodiment, the ITR sequences from AAV2, or the deleted version thereof (ΔITR), are used. However, ITRs from other AAV sources may be selected. Where the source of the ITRs is from AAV2 and the AAV capsid is from another AAV source, the resulting vector may be termed pseudotyped. Typically, an expression cassette for an AAV vector comprises an AAV 5′ ITR, the hATP7B coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used. In one embodiment, the 5′ ITR is that shown in SEQ ID NO: 14. In one embodiment, the 3′ ITR is that shown in SEQ ID NO: 15.

In one embodiment, the expression control sequences include one or more enhancer. In one embodiment, the En34 enhancer is included (34 bp core enhancer from the human apolipoprotein hepatic control region), which is shown in SEQ ID NO: 4. In another embodiment, the EnTTR (100 bp enhancer sequence from transthyretin) is included. Such sequence is shown in SEQ ID NO: 5. See, Wu et al, Molecular Therapy, 16(2):280-289, February 2008, which is incorporated herein by reference. In yet another embodiment, the α1-microglogulin/bikunin precursor enhancer is included. In yet another embodiment, the ABPS (shortened version of the 100 bp distal enhancer from the al-microglogulin/bikunin precursor [ABP] to 42 bp) enhancer is included. Such sequence is shown in SEQ ID NO: 6. In yet another embodiment, the ApoE enhancer is included. Such sequence is shown in SEQ ID NO: 7. In another embodiment, more than one enhancer is present. Such combination may include more than one copy of any of the enhancers described herein, and/or more than one type of enhancer.

Expression of the hATP7B coding sequence is driven from a liver-specific promoter. Because of the size of the ATP7B transgene, the use of promoter of relatively small size is desirable. An illustrative plasmid and vector described herein uses the modified thyroxine binding globulin (TBG-S1) promoter (SEQ ID NO: 8). In another embodiment, the TBG promoter is used. The TBG promoter sequence is shown in SEQ ID NO: 9. Alternatively, other liver-specific promoters may be used such as the transthyretin promoter (TTR promoter), as shown in SEQ ID NO: 11, or a modified transthyretin promoter (mTTR promoter), as shown in nt 21 to nt 190 of SEQ ID NO: 11. Another suitable promoter is the alpha 1 anti-trypsin (A1AT), or a modified version thereof (which sequence is shown in SEQ ID NO: 10. Various promoter and enhancer combinations are discussed in the examples below.

Other suitable promoters include human albumin (Miyatake et al., J. Virol., 71:5124 32 (1997)), humAlb; the Liver Specific promoter (LSP), and hepatitis B virus core promoter, (Sandig et al., Gene Ther., 3:1002-9(1996). Sec, e.g., The Liver Specific Gene Promoter Database, Cold Spring Harbor, rulai.schl.edu/LSPDrulai.schl.edu/LSPD, which is incorporated by reference. Although less desired, other promoters, such as viral promoters, constitutive promoters, regulatable promoters [see, e.g., WO 2011/126808 and WO 2013/04943], or a promoter responsive to physiologic cues may be used may be utilized in the vectors described herein.

In addition to a promoter, an expression cassette and/or a vector may contain other appropriate transcription initiation, termination, enhancer sequences, and efficient RNA processing signals. Such sequences include splicing and polyadenylation (polyA) signals; regulatory elements that enhance expression (e.g., WPRE); sequences that stabilize cytoplasmic mRNA; sequences that enhance translation efficiency (i.e., Kozak consensus sequence); sequences that enhance protein stability; and when desired, sequences that enhance secretion of the encoded product. In one embodiment, a KOZAK sequence is included. In one embodiment, a polyadenylation (polyA) signal is included to mediate termination of hATP7B mRNA transcripts. A polyA signal useful herein is an artificial polyA which is about 75 bp in size (PA75) shown in SEQ ID NO: 13. Examples of other suitable polyA sequences include, e.g., bovine growth hormone (SEQ ID NO: 12), SV40, rabbit beta globin, and TK polyA, amongst others.

In one embodiment, the regulatory sequences are selected such that the total rAAV vector genome is about 3.0 to about 5.5 kilobases in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. In a further embodiment, the total rAAV vector genome is about 5.1 kb or about 5.0 kb in size. The size of the vector genome may be manipulated based on the size of the regulatory sequences including the promoter, enhancer, intron, poly A, etc. See, Wu et al, Mol Ther, January 2010 18(1):80-6, which is incorporated herein by reference.

In one embodiment, the rAAV vector genome comprises nt 1 to nt 5134 of SEQ ID NO: 23, nt 1 to nt 5056 of SEQ ID NO: 24, nt 1 to nt 5064 of SEQ ID NO: 25, nt 1 to nt 5068 of SEQ ID NO: 26, nt 1 to nt 5048 of SEQ ID NO: 27, nt 1 to nt 4284 of SEQ ID NO: 28, nt 1 to nt 4677 of SEQ ID NO: 29, nt 1 to nt 4978 of SEQ ID NO: 30, nt 1 to nt 4876 of SEQ ID NO: 31, nt 1 to nt 4945 of SEQ ID NO: 32, nt 1 to nt 3628 of SEQ ID NO: 33, nt 1 to nt 4021 of SEQ ID NO: 34, or nt 1 to nt 4627 of SEQ ID NO: 35.

Exemplary production plasmids to generate rAAVs are shown in SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, and SEQ ID NO: 35.

### 5.1.2. Compositions

In one embodiment, the rAAV.hATP7B virus is provided in a pharmaceutical composition which comprises an aqueous carrier, excipient, diluent or buffer. In one embodiment, the buffer is PBS. In a specific embodiment, the rAAV.hATP7B formulation is a suspension containing an effective amount of rAAV.hATP7B vector suspended in an aqueous solution containing 0.001% Pluronic F-68 in TMN200 (200 mM sodium chloride, 1 mM magnesium chloride, 20 mM Tris, pH 8.0). However, various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

For example, a suspension as provided herein may contain both NaCl and KCl. The pH may be in the range of 6.5 to 8.5, or 7 to 8.5, or 7.5 to 8. A suitable surfactant, or combination of surfactants, may be selected from among Poloxamers, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension. In another embodiment, the vector is suspended in an aqueous solution containing 180 mM sodium chloride, 10 mM sodium phosphate, 0.001% Poloxamer 188, pH 7.3.

In one embodiment, the formulation is suitable for use in human subjects and is administered intravenously. In one embodiment, the formulation is delivered via a peripheral vein by bolus injection. In one embodiment, the formulation is delivered via a peripheral vein by infusion over about 10 minutes (±5 minutes). In one embodiment, the formulation is delivered via a peripheral vein by infusion over about 20 minutes (±5 minutes). In one embodiment, the formulation is delivered via a peripheral vein by infusion over about 30 minutes (±5 minutes). In one embodiment, the formulation is delivered via a peripheral vein by infusion over about 60 minutes (±5 minutes). In one embodiment, the formulation is delivered via a peripheral vein by infusion over about 90 minutes (±10 minutes). However, this time may be adjusted as needed or desired. Any suitable method or route can be used to administer an AAV-containing composition as described herein, and optionally, to co-administer other active drugs or therapies in conjunction with the AAV-mediated delivery of hATP7B described herein. Routes of administration include, for example, systemic, oral, inhalation, intranasal, intratracheal, intraarterial, intraocular, intravenous, intramuscular, subcutaneous, intradermal, and other parental routes of administration.

In one embodiment, the formulation may contain, e.g., about 1.0×10¹¹ genome copies per kilogram of patient body weight (GC/kg) to about 1×10¹⁴ GC/kg, about 5×10¹¹ genome copies per kilogram of patient body weight (GC/kg) to about 3×10¹³ GC/kg, or about 1×10¹² to about 1×10¹⁴ GC/kg, as measured by oqPCR or digital droplet PCR (ddPCR) as described in, e.g., M. Lock et al, Hum Gene Ther Methods. 2014 April; 25(2):115-25. doi: 10.1089/hgtb.2013.131. Epub 2014 Feb. 14, which is incorporated herein by reference. In one embodiment, the rAAV.hATP7B formulation is a suspension containing at least 1×10¹³ genome copies (GC)/mL, or greater, as measured by oqPCR or digital droplet PCR (ddPCR) as described in, e.g., M. Lock et al, supra.

In order to ensure that empty capsids are removed from the dose of AAV.hATP7B that is administered to patients, empty capsids are separated from vector particles during the vector purification process, e.g., using the method discussed herein. In one embodiment, the vector particles containing packaged genomes are purified from empty capsids using the process described in WO 2017/100676 and entitled “Scalable Purification Method for AAV8”, which is incorporated by reference herein. Briefly, a two-step purification scheme is described which selectively captures and isolates the genome-containing rAAV vector particles from the clarified, concentrated supernatant of a rAAV production cell culture. The process utilizes an affinity capture method performed at a high salt concentration followed by an anion exchange resin method performed at high pH to provide rAAV vector particles which are substantially free of rAAV intermediates. Similar purification methods can be used for vectors having other capsids.

While any conventional manufacturing process can be utilized, the process described herein (and in WO 2017/100676) yields vector preparations wherein between 50 and 70% of the particles have a vector genome, i.e., 50 to 70% full particles. Thus for an exemplary dose of 1.6×10¹² GC/kg, and the total particle dose will be between 2.3×10¹² and 3×10¹² particles. In another embodiment, the proposed dose is one half log higher, or 5×10¹² GC/kg, and the total particle dose will be between 7.6×10¹² and 1.1×10¹³ particles. In one embodiment, the formulation is be characterized by an rAAV stock having a ratio of “empty” to “full” of 1 or less, preferably less than 0.75, more preferably, 0.5, preferably less than 0.3.

A stock or preparation of rAAV8 particles (packaged genomes) is “substantially free” of AAV empty capsids (and other intermediates) when the rAAV8 particles in the stock are at least about 75% to about 100%, at least about 80%, at least about 85%, at least about 90%, at least about 95%, or at least 99% of the rAAV8 in the stock and “empty capsids” are less than about 1%, less than about 5%, less than about 10%, less than about 15% of the rAAV8 in the stock or preparation.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacylamide gels (e.g., Novex). Silver staining may be performed using SilverXpress (Invitrogen, CA) according to the manufacturer's instructions. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is provided herein which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

### 5.2 Patient Population

As discussed above, a subject having WD of any severity is the intended recipient of the compositions and methods described herein.

Subjects may be permitted to continue their standard of care treatment(s) (e.g., diet low in copper; treatment with chelating agents such as D-penicillamine and trientine. Other agents include sodium dimercaptosuccinate, dimercaptosuccinic acid, zinc, and tetrathiomolybdate) prior to and concurrently with the gene therapy treatment at the discretion of their caring physician. In the alternative, the physician may prefer to stop standard of care therapies prior to administering the gene therapy treatment and, optionally, resume standard of care treatments as a co-therapy after administration of the gene therapy.

Desirable endpoints of the gene therapy regimen would provide functional ATP7B enzyme resulting in a reduction of serum copper levels of 25% or more. In one embodiment, urinary copper excretion of 3-8 μmol or less per 24 hours is desirable.

Many tests can be used to investigate patients who may have Wilson disease, including non-ceruloplasmin-bound copper (NCC; also called the “free copper” or copper index), 24-h urine copper, hepatic copper, and genetic mutation testing. Methods for measurement of copper levels are known in the art e.g, as described by McMillin et al, Am J Clin Pathol. 2009; 131(2):160-165, which is incorporated herein by reference. In one embodiment, patients achieve desired circulating ATP7B levels after treatment with rAAV.hATP7B, alone and/or combined with the use of adjunctive treatments.

### 5.3. Dosing & Route of Administration

In one embodiment, the rAAV.hATP7B vector is delivered as a single dose per patient. In one embodiment, the subject is delivered the minimal effective dose (MED) (as determined by preclinical study described in the Examples herein). As used herein, MED refers to the rAAV.hATP7B dose required to provide functional ATP7B enzyme resulting in a reduction of serum copper levels of 25% or more.

As is conventional, the vector titer is determined on the basis of the DNA content of the vector preparation. In one embodiment, quantitative PCR or optimized quantitative PCR as described in the Examples is used to determine the DNA content of the rAAV.hATP7B vector preparations. In one embodiment, digital droplet PCR as described above is used to determine the DNA content of the rAAV.hATP7B vector preparations. In one embodiment, the dosage is about 1×10¹¹ genome copies (GC)/kg body weight to about 1×10¹³ GC/kg, inclusive of endpoints. In one embodiment, the dosage is 5×10¹¹ GC/kg. In another embodiment, the dosage is 5×10¹² GC/kg. In specific embodiments, the dose of rAAV.hATP7B administered to a patient is at least 5×10¹¹ GC/kg, 1×10¹² GC/kg, 1.5×10¹² GC/kg, 2.0×10¹² GC/kg, 2.5×10¹² GC/kg, 3.0×10¹² GC/kg, 3.5×10¹² GC/kg, 4.0×10¹² GC/kg, 4.5×10¹² GC/kg, 5.0×10¹² GC/kg, 5.5×10¹² GC/kg, 6.0×10¹² GC/kg, 6.5×10¹² GC/kg, 7.0×10¹² GC/kg, or 7.5×10¹² GC/kg. Also, the replication-defective virus compositions can be formulated in dosage units to contain an amount of replication-defective virus that is in the range of about 1.0×10⁹ GC to about 1.0×10¹⁵ GC. As used herein, the term “dosage” can refer to the total dosage delivered to the subject in the course of treatment, or the amount delivered in a single (of multiple) administration.

In some embodiments, rAAV.hATP7B is administered in combination with one or more therapies for the treatment of WD, such as a low copper diet or administration of D-penicillamine, trientine, sodium dimercaptosuccinate, dimercaptosuccinic acid, zinc, and/or tetrathiomolybdate.

### 5.4. Measuring Clinical Objectives

Measurements of efficacy of treatment can be measured by transgene expression and activity as determined by ATP7B activity and or non-ceruloplasmin-bound copper (NCC; also called the “free copper” or copper index), 24-h urine copper, or hepatic copper levels. Further assessment of efficacy can be determined by clinical assessment of dietary copper tolerance.

As used herein, the rAAV.hATP7B vector herein “functionally replaces” or “functionally supplements” the patients defective ATP7B with active ATP7B when the patient expresses a sufficient level of ATP7B to achieve ATP7B activity resulting a 25% or greater reduction in non-ceruloplasmin-bound copper, 24-h urine copper, and/or hepatic copper.

The following examples are illustrative only and are not intended to limit the present invention.

### EXAMPLES

The following examples are illustrative only and are not intended to limit the present invention.

### Example 1: AAV Vectors Containing hATP7B (AAV.hATP7Bco)

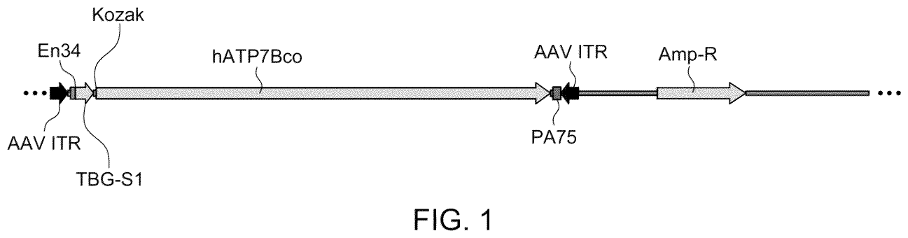

An exemplary gene therapy vector AAV8.En34.TBG-S1.hATP7Bco.PA75 was constructed by an AAV8 vector bearing a codon-optimized human hATP7B cDNA (hATP7Bco) under the control of a TBG-S1 promoter and an En34 enhancer (FIG. 1). The ATP7B expression cassette was flanked by AAV2 derived inverted terminal repeats (ITRs) and further included a Kozak consensus sequence and a PA75 poly(A) signal. The sequence of AAV8.En34.TBG-S1.hATP7Bco.PA75 genome is shown in nt 1 to nt 5056 of SEQ ID NO: 24.

The vector AAV8.En34.TTR.hATP7Bco.PA75 was constructed as described above with a hepatocyte specific TTR promoter instead of TBG-S1. The sequence of AAV8.En34.TTR.hATP7Bco.PA75 is shown in nt 1 to nt 5068 of SEQ ID NO: 26.

A modified TTR promoter with the sequence shown as nt 21 to nt 190 of SEQ ID NO: 11, instead of TBG-S1 was utilized to construct the vector AAV.En34.mTTR.hATP7Bco.PA75. The sequence of AAV.En34.mTTR.hATP7Bco.PA75 is shown in nt 1 to nt 5048 of SEQ ID NO: 27.

The ATP7B expression cassette of AAV8.EnTTR.TTR.hATP7Bco.PA75 vector was driven by an EnTTR enhancer and a TTR promoter with a Kozak consensus sequence and a PA75 poly(A) signal. The sequence of AAV8.EnTTR.TTR.hATP7Bco.PA75 is shown in nt 1 to nt 5134 of SEQ ID NO: 23.

The AAV8.EnABPS.TBG-S1.hATP7Bco.PA75vector encodes a codon-optimized human ATP7B cDNA (hATP7Bco) under the control of an ABP-S2 (ABPS enhancer) enhancer and a TBG-S1 promoter, with a Kozak consensus sequence and a PA75 poly (A) signal. The sequence of AAV8.ABPS.TBG-S1.hATP7Bco.PA75 is shown in nt 1 to nt 5064 of SEQ ID NO: 25.

Additionally, truncated hATP7Bco vectors, including AAV8.En34.TBG-S1.hATP7Bco (MBD1Del).PA75 (as shown in nt 1 to nt 4945 of SEQ ID NO: 32), AAV8.En34.TBG-S1.hATP7Bco (MBD2Del).PA75 (as shown in nt 1 to nt 4876 of SEQ ID NO: 31), AAV8.En34.TBG-S1.hATP7Bco (MBD3Del).PA75 (nt 1 to nt 4978 of SEQ ID NO: 30), AAV8.En34.TBG-S1.hATP7Bco (MBD1-2Del).PA75 (nt 1 to nt 4627 of SEQ ID NO: 35), AAV8.En34.TBG-S1.hATP7Bco (MBD1-4Del).PA75 (nt 1 to nt 4021 of SEQ ID NO: 34), AAV8.En34.TBG-S1.hATP7Bco (MBD1-5Del).PA75 (nt 1 to nt 3628 of SEQ ID NO: 33), AAV8.TBG.hATP7Bco (MBD1-4Del).PA75 (nt 1 to nt 4677 of SEQ ID NO: 29), and AAV8.TBG.hATP7Bco (MBD1-5Del).PA75 (nt 1 to nt 4284 of SEQ ID NO: 28) were designed, constructed and produced as the indicated truncated hATP7Bco with a Kozak consensus sequence, a PA75 Ploy (A) signal and promoters and enhancers as shown in Table 2.

Briefly, plasmids expressing a codon-optimized version of hATP7B (hATP7Bco) from a reduced sized transthyretin enhancer and promoter were packaged with the AAV8 viral capsid.

The vector was prepared using conventional triple transfection techniques in 293 cells as described e.g., by Mizukami, Hiroaki, et al. A Protocol for AAV vector production and purification. Diss. Division of Genetic Therapeutics, Center for MolecularMedicine, 1998., which is incorporated herein by reference. All vectors were produced by the Vector Core at the University of Pennsylvania as previously described [Lock, M., et al, Hum Gene Ther, 21: 1259-1271 (2010)].

TABLE 2

| | | | | | Age of | Difference | Percentage | Difference | Percentage |
|---|---|---|---|---|---|---|---|---|---|
| | | | Size | Mouse | mice at | in serum | difference | in serum | difference |
| | | | ITR- | sex | study | copper | in serum | copper | in serum |
| Key elements in the vector | | | ITR | (female | initiation | levels from | copper from | levels from | copper from |
| Enhancer | Promoter | Transgene | (bp) | or male) | (weeks) | W0 to W2 | W0 to W2 | W0 to W4 | W0 to W4 |
| EnTTR | TTR | hATP7Bco | 5134 | M | 12 | 0.150 | 31% | 0.158 | 33% |
| En34 | TBG-S1 | hATP7Bco | 5056 | M | 11 | 0.182 | 38% | 0.252 | 52% |
| En34 | TTR | hATP7Bco | 5069 | M | 10 | 0.120 | 25% | 0.176 | 36% |
| ABPS | TBG-S1 | hATP7Bco | 5064 | M | 9 | 0.134 | 28% | 0.154 | 32% |
| En34 | mTTR | hATP7Bco | 5084 | M | 12 | 0.294 | 61% | 0.232 | 48% |
| En34 | TBG-S1 | hATP7Bco MBD1 Del | 4945 | M | 11 | 0.106 | 22% | 0.200 | 41% |
| En34 | TBG-S1 | hATP7Bco MBD2 Del | 4876 | M | 11 | 0.052 | 11% | 0.062 | 13% |
| En34 | TBG-S1 | hATP7Bco MBD3 Del | 4978 | M | 10 | 0.058 | 12% | 0.076 | 16% |
| En34 | TBG-S1 | hATP7Bco MBD1-2 Del | 4627 | M | 11 | 0.072 | 15% | 0.106 | 22% |
| En34 | TBG-S1 | hATP7Bco MBD1-4 Del | 4021 | M | 11 | 0.132 | 27% | 0.240 | 50% |
| En34 | TBG-S1 | hATP7Bco MBD1-5 Del | 3628 | M | 10 | 0.190 | 39% | 0.302 | 63% |

| | | | | | | | | |
|---|---|---|---|---|---|---|---|---|
| Full TBG | hATP7Bco MBD1-4 Del | 4677 | M | 10 | 0.160 | 33% | 0.206 | 43% |
| Full TBG | hATP7Bco MBD1-5 Del | 4284 | M | 10 | 0.300 | 62% | 0.380 | 79% |

### Example 2: A Mouse Model of Wilson's Disease

Prior to the development of gene therapeutic approaches for the treatment of Wilson's disease, the animal model of the disease phenotype must be fully characterized. The studies described herein are the first detailed characterization of both the tx^{J} mouse strain and the evaluation of copper metabolism and disease pathology following fostering of all Atp7b KO mice from birth. In the absence of the conflicting copper deficiency provided prior to weaning due to the Atp7b deficiency in the mammary glands of diseased mothers, the time line of disease progression was accurately determined. Atp7b KO mice accumulate copper in the liver from birth with severe copper accumulation evident by two months of age with concurrent liver disease.

Monogenic diseases affecting single organs are attractive targets for gene therapy approaches, especially if there is relatively little associated histologic lesions. However, for metabolic disorders affecting the liver, there can often be severe damage to the liver parenchyma as a result of the disease. One of the classical examples of this is Wilson's disease, an autosomal, recessive disease caused by mutations in the Wilson's disease protein (a copper-transporting P-type ATPase, Atp7b). Lack of functional Atp7b results in the accumulation of copper in the liver and other tissues, which manifests as liver disease with neurological or psychiatric symptoms. Wilson's disease can be treated by reducing copper absorption or removing excess copper from the body using chelation therapy, but, as for many other metabolic disorders, liver transplantation can both correct the genetic deficiency associated with disease and replace a dysfunctional organ.

Wilson's disease affects 1:30,000 people with different disease symptoms and progression. To enable the development of new therapeutic alternatives to chelation and liver transplantation, a reliable animal model of this disease must be fully characterized. There are several previously reported rat and mouse animal models of Wilson's disease, including the Long-Evans Cinnamon (LEC) rats and various transgenic mouse strains (1-6). For evaluation of the Wilson's disease phenotype in a mouse model, we selected the toxic milk mouse (tx^{J}) available from Jackson Labs (2). These mice have a Gly712Asp missense mutation in the Atp7b gene, which is located in the second putative membrane-spanning domain of the encoded protein and results in a dysfunctional Atp7b protein.

The original name for this strain, the toxic milk mouse, is a result of Atp7b also being expressed in mammary tissue in addition to liver. Therefore, the “spontaneous arising” of the toxic milk phenotype in this model is a direct result of Wilson's disease (7). Due to the deficiency in the Atp7b protein, copper cannot be transported from the mother into breastmilk (8). Diseased pups that suckle from diseased mothers will present with an inverse form of Wilson's disease (copper deficiency) as they are unable to receive any copper in their diet until they are weaned and consume normal mouse chow. This inverse form of Wilson's disease also accounts for the previously described white coat color and mental defects (9). Here, we prefer to describe this mouse model as an Atp7b knockout (KO), and not as the tx^{J}, due to the dysfunctional Atp7b protein and the fact that all mice used in these studies were fostered onto Balb/c foster mothers within 72 hours of birth to mitigate the toxic milk issue. Therefore, characterization of the Wilson's disease phenotype described here has been separated from any underlying issues related to copper deficiency prior to weaning.

All animal procedures were performed in accordance with protocols approved by the Institutional Animal Care and Use Committee (IACUC) of the University of Pennsylvania.

ATP7B KO mice do not express functional copper-transporting ATPase (Lutsenko et al, Function and Regulation of Human Copper-Transporting ATPases, Physiological Reviews, 87(3):1011-46 (July 2007), thus served as a mouse model for Wilson's disease. A natural history study was performed to evaluate the progression of Wilson's disease in ATP7B KO mice. The study was performed in two phases. In the first, the mice were necropsied at different ages for evaluation of liver disease. In the second phase, mice were followed to 9 months of age for evaluation of several biomarkers.

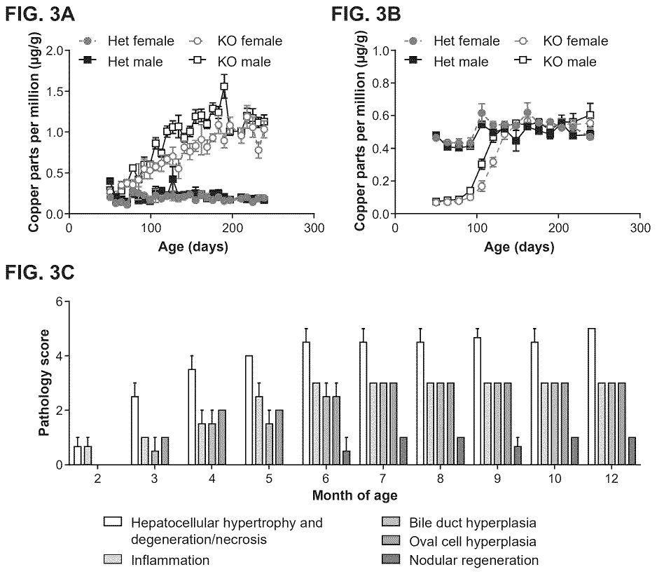

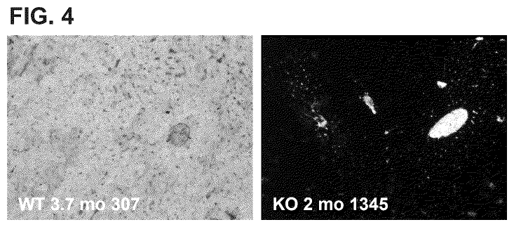

Atp7b heterozygous and KO mice were evaluated for urine and serum copper levels on a weekly and biweekly basis, respectively, from 2 months of age for 7 months (FIGS. 3A and 3B). Atp7b in the liver exports copper into the bile for excretion in feces (7). In the absence of Atp7b, excretion of copper will occur via the urinary tract. Urine copper levels were initially similar in both genotypes studied, but at 3 months of age the level of copper in the urine of the Atp7b KO mice started to increase compared to the heterozygous animals (FIG. 3A). At the completion of the study urine copper levels averaged 0.18 μg/g in heterozygous mice and 1.08 μg/g in Atp7b KO mice. Serum copper levels in the Atp7b KO mice at two months of age were significantly different from heterozygous animals at 0.07 μg/g compared to 0.47 μg/g (p<0.0001 by Student's t test, FIG. 3B). At 3-4 months of age, serum copper levels in the Atp7b KO mice began to rise, reaching levels equivalent to that of the heterozygous mice. There were little to no differences in either urine or serum copper levels in male or female mice. The results of the first phase show that accumulation of copper in the liver was observed from birth. When the Atp7b KO mice reached 2 month-old, Timm's copper stain revealed a severe copper accumulation in the liver (FIG. 4).

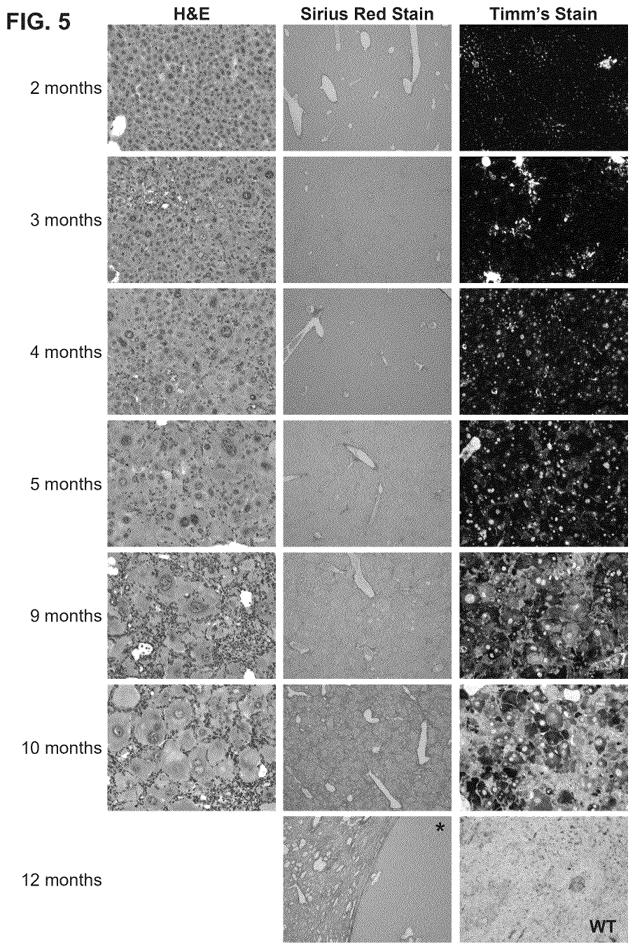

Monogenic diseases affecting liver metabolism can be divided into two subcategories, those with liver lesions and those without. Wilson's disease patients do exhibit moderate to severe liver disease, usually manifesting as cirrhosis (13, 14). Therefore, we wanted to evaluate the time course for development of liver pathology in this mouse model of Wilson's disease. Atp7b KO mice were necropsied at 2, 3, 4, 5, 9, 10, and 12 months of age for evaluation of liver lesions over time (FIG. 5). Sections of the liver were stained with H&E, and evaluated for histopathology according to the 1-5 scoring system (FIG. 3C). Minimal hepatocellular hypertrophy and degeneration along with single cell necrosis and inflammation were present in Atp7b KO mice from two months of age. The severity of hepatocellular hypertrophy, degeneration and necrosis was highest by 6 months of age and remained consistent thereafter. Other parameters evaluated, including inflammation, bile duct hyperplasia, and oval cell hyperplasia, increased over time from 2-3 months to 7 months of age. There was a concurrent rise in the liver transaminases, ALT and AST, from normal values at two months of age to 199 U/l and 381 U/l for ALT and AST, respectively, at 10 months (Table 3). The extent of liver damage at 10 months of age was also evident from the serum total bilirubin levels as these suddenly spiked at 2.5 mg/dl. In addition to the liver lesions, areas of hepatic nodular regeneration were evident in this mouse model from 6 months of age (an example is shown by the asterisk in FIG. 5). This is perhaps unsurprising due to the severity of the liver disease.

TABLE 3

| | | | | Total | |
|---|---|---|---|---|---|
| | Age | ALT | AST | bilirubin | |
| | (months) | (U/l) | (U/l) | (mg/dl) | n |
| ATP7B KO | 2 | 16 | 30 | 0.2 | 1 |
| | 3 | 164 | 190 | 0.2 | 1 |
| | 4 | 174 | 224 | 0.3 | 2 |
| | 5 | 279 | 175 | 0.2 | 5 |
| | 9 | 152 | 266 | 0.6 | 18 |
| | 10 | 199 | 381 | 2.5 | 2 |
| WT | 2 | 28 | 50 | 0.2 | 13 |
| | 3 | 29 | 54 | 0.2 | 8 |
| | 4 | 49 | 39 | 0.2 | 4 |
| | 5 | 30 | 41 | 0.1 | 2 |

Liver sections were also evaluated for fibrosis and copper accumulation by Sirius Red and Timm stain, respectively (FIG. 5). Focal or multifocal periportal fibrosis was seen by 3 months of age in the Atp7b KO mice. This rapidly progressed to diffuse bridging fibrosis with architectural disruption by 7 months of age. The development of liver lesions was likely due to the massive accumulation of copper in the liver as seen by the Timm's stain (FIG. 5). From two months of age, the liver of Atp7b KO mice had become saturated with copper as shown by the black staining. Accumulation of copper in the liver occurs due to disrupted export of copper into the bile in the absence of functional Atp7b. The levels of copper in the liver decreased over time, likely due to hepatocyte damage and release of copper into the serum. This has been previously shown, where copper values are significantly reduced with submassive or massive necrosis, and reduced even further with regeneration and fibrosis (1, 15-17). The fibrous connective tissue and regenerating hepatocytes do not contain excess copper concentration (18).

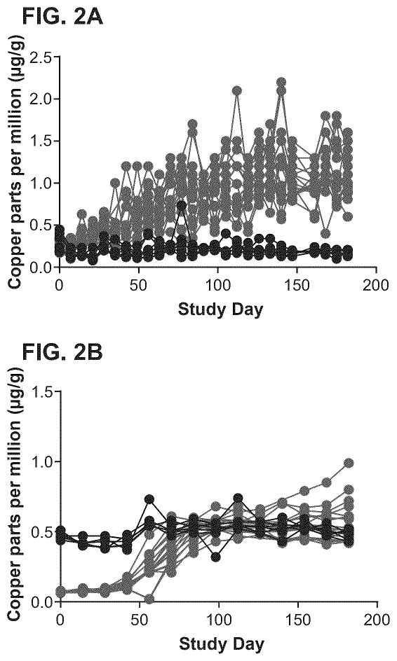

The copper levels in the urine of ATP7B KO mice were also monitored. Samples were collected weekly in the natural history study and then inductively coupled plasma-mass spectrometry was performed to assess the copper concentration. The results demonstrate that while the heterozygous littermates maintained a low copper level, Atp7b KO mice exhibited an overflow of copper into urine starting at about the third week followed by a steady growth of the urinary concentration of copper over the observation period (FIG. 2A, 3A), representing the high urinary copper excretion rate observed in the Wilson's disease patients.

The serum of Atp7b KO mice was collected biweekly to assess the copper concentration via inductively coupled plasma-mass spectrometry. The result showed a very low serum copper level until 2 months of age compared to heterozygous littermates suggesting a compromised ability of extracting copper from the tissue to the blood in the Atp7b KO mice. When the Atp7b KO mice were 3 to 4 month old, the serum concentration of copper significantly increased and reached the level of the heterozygous littermates (FIG. 2B, 3B).

In this Atp7b KO mouse model, the progression of disease was as follows: Severe copper accumulation in the liver was seen by Timm's copper stain at two months of age, but did decrease over time similar to that previously described by others (1, 15-17). Due to fostering of the pups, copper is likely accumulating in the liver of Atp7b KO mice from birth and reaching saturation levels around 2-3 months of age. Following development of liver disease at two months of age, copper is likely released into the serum, resulting in an apparent decrease in accumulation in hepatocytes and rising serum copper levels by 3-4 months of age. This rise in serum copper levels from initially low levels to those similar to heterozygous mice is different to what has been reported previously for another mouse model of Wilson's disease (17), where serum copper levels in the Atp7b−/− strain are similar to that seen in wild type mice at 6 weeks of age and increase over time to 2 to 3-fold higher than wild type mice by 44 weeks of age.

We observed that the overflow of copper into urine starts at ˜3 months of age, which similarly could be due to hepatocellular necrosis and subsequent release of accumulated copper. Alternative mechanisms for excretion of copper via the kidney have been suggested, including lack of Atp7b activity in the kidney resulting in increased excretion (23-25), or accumulation of copper in the liver leading to downregulation of the liver copper transporter, Ctrl, and urinary excretion by a small copper carrier (26). Again, the time course of urinary copper excretion differs in this mouse model to that reported previously. Here, urinary copper excretion is initially similar in heterozygous and Atp7b KO mice, but increases in the KO mice over the course of the study. In comparison, urinary copper levels are 3-fold greater than wild type mice at 6 weeks of age in the Atp7b−/− strain, increase up until 14-20 weeks old, and then decrease substantially at 20 weeks of age (26).

Hepatocellular hypertrophy, degeneration and necrosis peak at 6 months of age with likely concomitant observation of areas of hepatic nodular regeneration from this age onwards. This progression towards regeneration has been reported for other mouse models of Wilson's disease (17). However, unlike in the other mouse models, there was no evidence of cholangiocarcinoma in the Atp7b KO mouse described here. Regions of fibrosis were seen by 3 months of age, which rapidly increased in severity over time with architectural disruption by 7 months of age. While there are marked increases in serum transaminases by 3-4 months of age, increases in serum total bilirubin levels only start to occur at 9 months, indicating advanced liver disease.

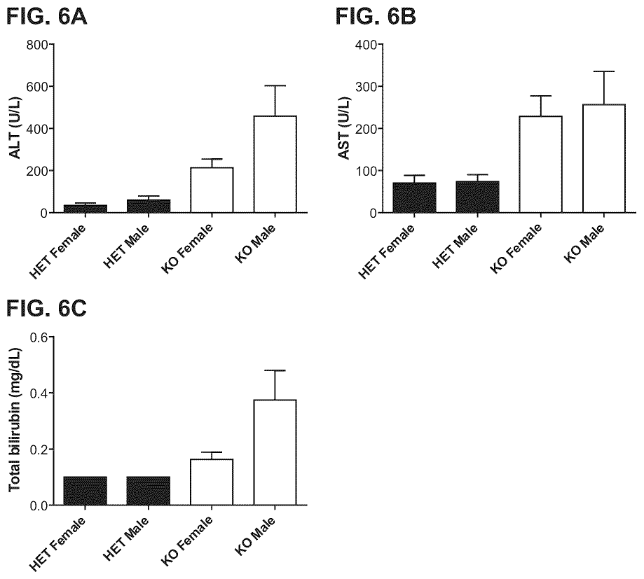

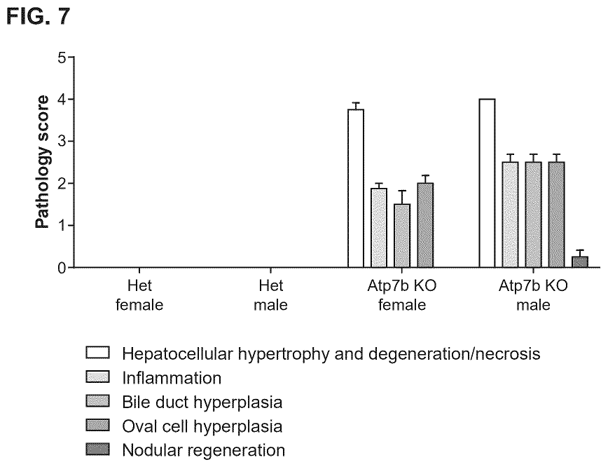

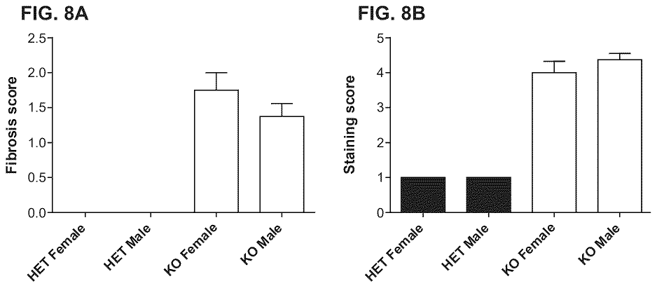

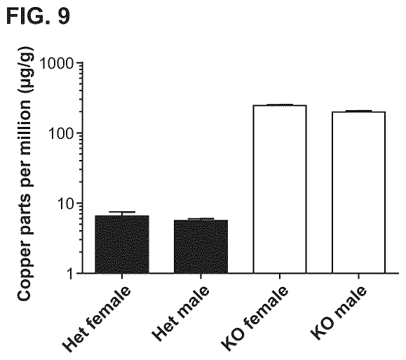

In the second phase of the natural history study, Atp7b KO and heterozygous littermates were sacrificed at 8 months of age. ALT, AST and total bilirubin levels were obtained. FIG. 6A-6C. H&E stain and then histopathology evaluated by pathologist against a scoring scheme, which is shown in FIG. 7. Fibrosis score and copper staining score by Timm's Stain are shown in FIGS. 8A and 8B, respectively. Copper levels in the liver are shown in FIG. 9.

### Example 3: AAV8.hATP7Bco Vectors in the Model of Wilson's Disease

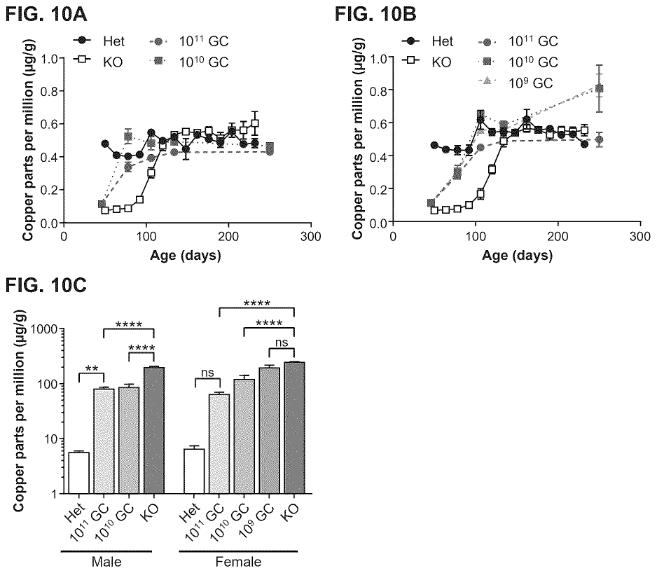

Male Atp7b KO mice were injected IV with 10¹⁰ GC/mouse and 10¹¹ GC/mouse of AAV8.TTR.hATP7Bco, and female Atp7b KO mice were injected IV with 10⁹, 10¹⁰, and 10¹¹ GC/mouse of the same vector. Serum copper levels were monitored following vector injection (FIGS. 10A and 10B). Administration of 10¹⁰ or 10¹¹ GC/mouse in male Atp7b KO mice increased serum copper levels from an average of 0.11 μg/g to 0.52 μg/g and 0.34 μg/g by two weeks post vector administration, respectively (FIG. 10A). However, there was a lesser effect in female mice (FIG. 10B). Mice were sacrificed at 7 months post vector administration at ˜9 months of age, and liver was harvested for evaluation of liver copper levels. Liver copper levels in heterozygous and Atp7b KO mice averaged 6 μg/g and 222 μg/g, respectively (FIG. 10C). AAV8 vector administration at doses>10⁹ GC/mouse resulted in significant decreases in liver copper levels compared to age-matched, uninjected Atp7b KO mice. However, there were no significant differences between liver copper levels in female mice administered with 10⁹ GC/mouse and control Atp7b KO mice. For this measurement, there was a stronger effect of the high vector dose in female mice as liver copper levels were not significantly different from heterozygous mice (FIG. 10C).

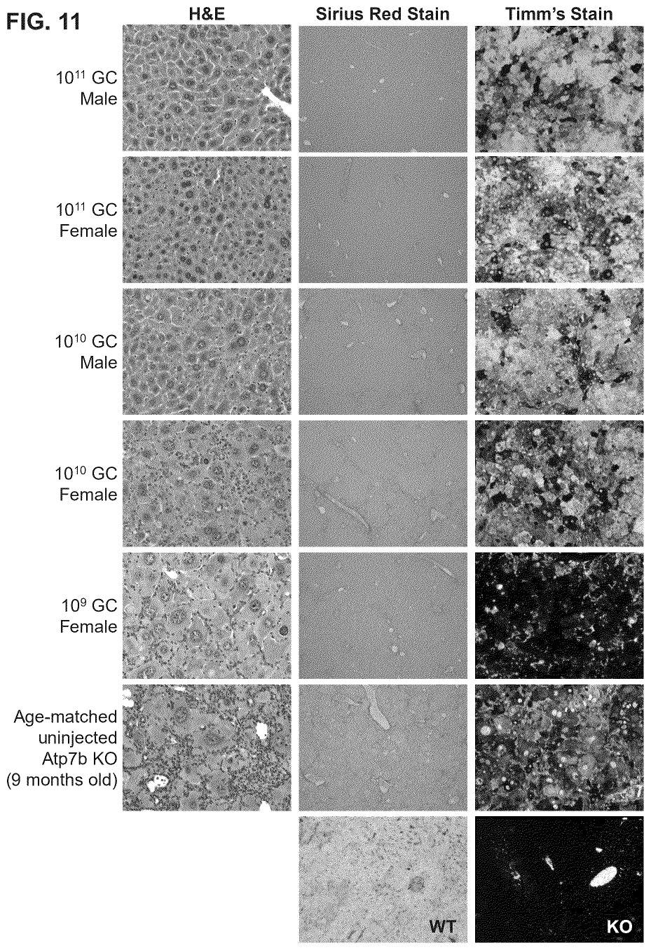

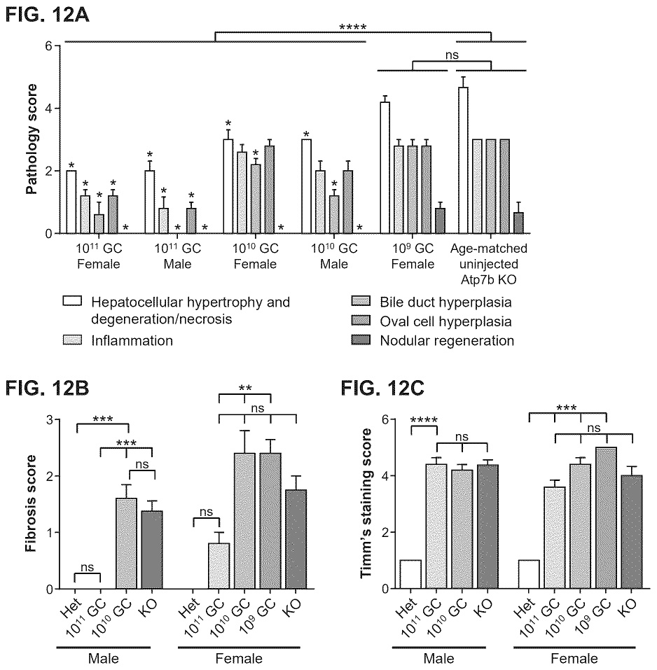

Mice administered with AAV8 vector were necropsied at 9 months of age, and liver was histologically evaluated for parameters of copper-associated liver disease, including fibrosis and copper levels by Timm's stain (FIG. 11). Sections of the liver were stained with H&E and histologically evaluated according to the 1-5 scoring system (FIG. 12A). Similar to that seen for liver copper levels, there was no significant difference in liver lesions between age-matched, uninjected Atp7b KO mice and female mice administered with 10⁹ GC/mouse of AAV8 vector. There was a dose-dependent decrease in liver lesions with male mice injected with 10¹¹ GC/mouse, which were observed to have only mild karyocytomegaly (denoting hepatocellular hypertrophy and degeneration), mild inflammation, and focal or multifocal periportal oval cell hyperplasia. There was a significant reduction in hepatocellular hypertrophy, degeneration/necrosis and as well as bile duct hyperplasia compared to the age-matched, uninjected Atp7b KO mice in mice administered with 10¹⁰ and 10¹¹ GC/mouse (p<0.05). For inflammation and oval cell hyperplasia, significant reduction compared to age-matched, uninjected Atp7b KO mice was only observed at a dose of 10¹¹ GC/mouse (p<0.05). When the histopathologic parameters were combined and evaluated using Fisher's combined probability test, there was a significant difference compared to age-matched, uninjected Atp7b KO mice following administration of both 10¹⁰ and 10¹¹ GC/mouse (p<0.0001).

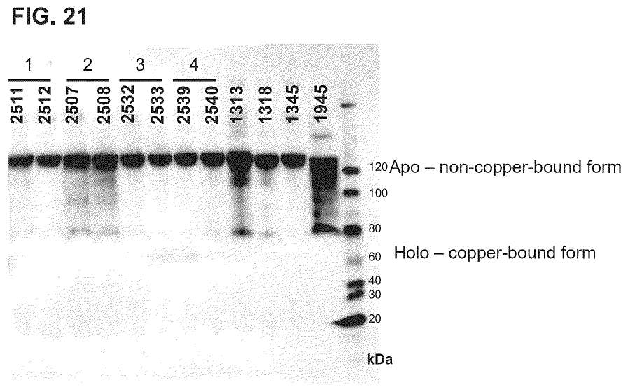

Male mice that received the highest vector dose also did not have fibrosis, which was evaluated with Sirius Red staining (FIGS. 11 and 12B). For all other vector-administered mice, there was no significant difference in fibrosis compared to age-matched, uninjected Atp7b KO mice (FIG. 12B). However, the reduction in fibrosis seen in female mice administered with 10¹¹ GC/mouse was sufficient that there was also no significant difference between liver fibrosis score in these mice and the female Atp7b heterozygous mice. Timm's staining for copper accumulation in the liver demonstrated similar results to that seen by the quantitative liver copper levels determined by inductively coupled plasma-mass spectrometry (FIG. 12C). There were no significant differences in Timm's staining score between age-matched, uninjected Atp7b KO mice and any of the vector-administered groups. To evaluate the efficacy of AAV8.hATP7Bco vectors, Atp7b KO mice received an i.v. administration of various gene therapy vectors at 3×10¹² GC/kg, including AAV8.EnTTR.TTR.hATP7Bco.PA75 (FIG. 21, group 1), AAV8.En34.TBG-S1.hATP7Bco.PA75 (FIG. 21, group 2), AAV8.En34.TTR.hATP7Bco.PA75 (FIG. 21, group 3) and AAV8.EnABPS.TBG-S1.hATP7Bco.PA75 (FIG. 21, group 4). Heterozygous and wild type littermates and Atp7b KO mice without treatment served as controls. Blood samples were collected weekly to evaluate the levels of both copper bound and non-copper-bound forms of ceruloplasmin via Western blot. On day 21 after the administration, appearance of copper-bound ceruloplasmin was observed in the Atp7b KO mice treated with AAV8.En34.TBG-S1.hATP7Bco.PA75, indicating a promoted copper extraction into blood (FIG. 21, group 2). However, under the experimental setting described above, the other three tested vectors did not display an increase in copper-bound ceruloplasmin (FIG. 21).

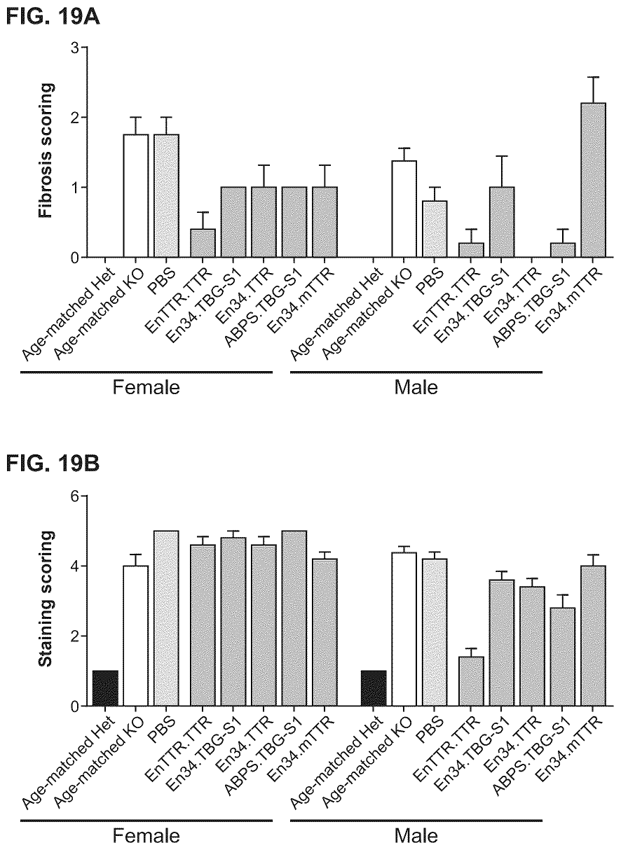

To assess the copper accumulation in the liver of the ATP7B KO mice injected with the AAV8.hATP7Bco vectors described herein, Timm's copper stain was performed on the liver sections thereof. Black deposits indicate copper accumulation. The results are shown in FIG. 19B. Injection with all five vectors to male Atp7b KO mice led to a decrease in the black deposits of the liver sections compared to the samples from non-treated or PBS-only mice while the AAV8.EnTTR.TTR.hATP7Bco.PA75 vector demonstrated the least black deposits and a pattern similar to wild type (FIG. 19B). Female Atp7b KO mice (FIG. 19B) demonstrated more copper deposits in liver compared to the male, indicating a gender difference. Still compared to non-treated mice, the female Atp7b KO mice demonstrated less copper deposits in liver. Generation of antibodies against human ATP7B protein in both male and female Atp7b KO mice injected with AAV.hATP7Bco vectors described herein is under investigation.

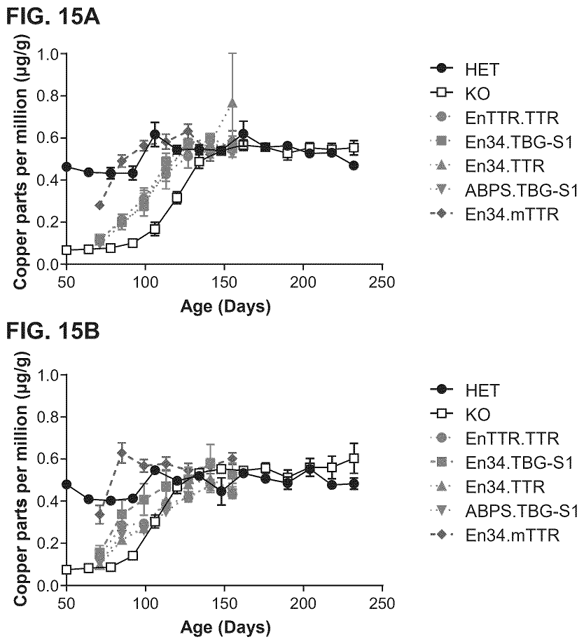

Meanwhile, the serum samples were collected and the copper concentrations were assessed as described in Example 2. The data was plotted in FIGS. 15A and 15B for female and male mice, respectively. The result demonstrates that the treatment of 3×10¹² GC/kg of the AAV8.hATP7Bco vectors described herein successfully increased the serum copper level in the Atp7b KO mice (FIGS. 15A and 15B).

Further tests were performed as follows:

| | | | | | Age of mice |
|---|---|---|---|---|---|
| | | Dose | | No. of | at study |
| Cohort | Vector | (GC/kg) | Sex | mice | initiation |
| 1 | EnTTR.TTR | 3.0 × 10¹² | F | 5 | 11 weeks |
| 2 | EnTTR.TTR | 3.0 × 10¹² | M | 5 | 12 weeks |
| 3 | En34.TBG-S1 | 3.0 × 10¹² | F | 5 | 11 weeks |
| 4 | En34.TBG-S1 | 3.0 × 10¹² | M | 5 | 11 weeks |
| 5 | En34.TTR | 3.0 × 10¹² | F | 5 | 10 weeks |
| 6 | En34.TTR | 3.0 × 10¹² | M | 5 | 10 weeks |
| 7 | APBS.TBG-S1 | 3.0 × 10¹² | F | 5 | 9 weeks |
| 8 | APBS.TBG-S1 | 3.0 × 10¹² | M | 5 | 9 weeks |
| 1 | En34.mTTR | 3.0 × 10¹² | F | 5 | 12 weeks |
| 2 | En34.mTTR | 3.0 × 10¹² | M | 5 | 12 weeks |

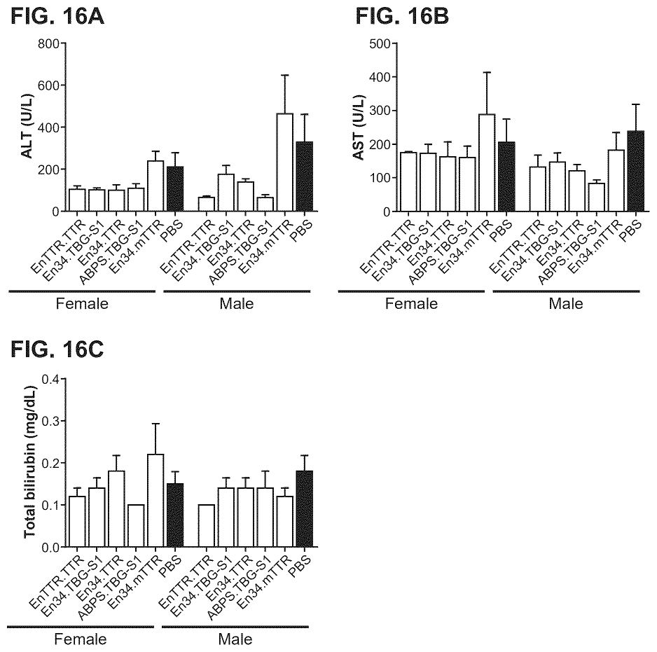

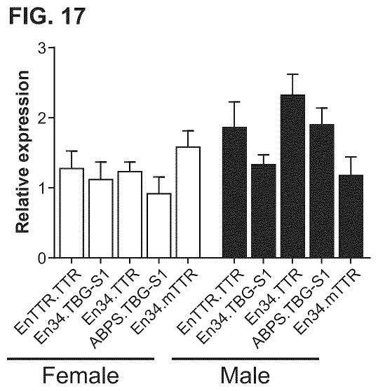

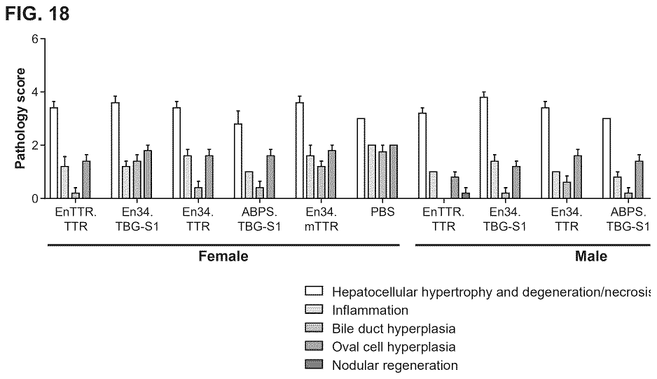

Serum copper levels over time are shown in FIGS. 15A and 15B. ALT, AST and total bilirubin levels were obtained. FIG. 16A-C. Relative ATP7B expression is shown in FIG. 17. H&E stain and then histopathology evaluated by pathologist against a scoring scheme, which is shown is FIG. 18. Fibrosis score and copper staining score by Timm Stain are shown in FIGS. 19A and 19B respectively.

### Example 4: Oxidase Activity of Ceruloplasmin in the Model of Wilson's Disease Injected with AAV.hATP7Bco Vectors

Copper is a potentially toxic metal but it is essential for a wide number of physiological functions acting as a co-factor of a variety of enzymes. After its intestinal absorption, copper is transported to hepatocytes where it binds to ATP7B located in the membrane of the trans-Golgi network (TGN). This large transmembrane protein is in charge of transferring the metal to copper-dependent enzymes. Loading of copper into ceruloplasmin is essential for the ferroxidase activity of this enzyme and constitutes an important secretory pathway for the metal, as 95% of copper present in the plasma of healthy individuals is bound to ceruloplasmin. Please see, e.g. Murillo, Oihana, et al. “Long-term metabolic correction of Wilson's disease in a murine model by gene therapy” Journal of Hepatology 64.2 (2016): 419-426, which is incorporated by reference herein.

To measure the oxidase activity, 20 μl of serum was processed using the Sigma Ceruloplasmin Activity Colorimetric Kit (MAK177) or the BioVision Ceruloplasmin Activity Colorimetric Kit following the protocols shown in the corresponding Product Information thereof, which is incorporated by reference herein. Oxidase activity assays were also performed according to the protocols shown in Schosinsky et al, “Measurement of ceruloplasmin from its oxidase activity in serum by use of o-dianisidine dihydrochloride” Clinical Chemistry 20.12 (1974): 1556-1563 and Murillo et al. “Long-term metabolic correction of Wilson's disease in a murine model by gene therapy” Journal of Hepatology 64.2 (2016): 419-426, which are incorporated by reference herein.

The oxidase activity of serum ceruloplasmin of the Atp7b KO mice was measured using the four assays described above. Samples from wildtype and heterozygous mice were served as control. No differences between wildtype and the Atp7b KO mice were detected.

Furthermore, Atp7b KO mice were treated with or without ammonium sulphate. Wildtype mice were provided as a control. No difference in the ceruloplasmin oxidase activity assay was detected between the wildtype or Atp7b KO mice using the protocol shown in Schosinsky et al, “Measurement of ceruloplasmin from its oxidase activity in serum by use of o-dianisidine dihydrochloride” Clinical Chemistry 20.12 (1974): 1556-1563.

In another experiment, Atp7b KO mice were treated with or without copper sulphate. Wildtype mice were provided as a control. The ceruloplasmin oxidase activity assay is performed to detect differences between the mice treated with or without copper sulphate using the four methods described above. Liver homogenates of the mice described herein are also collected and tested for oxidase activity of ceruloplasmin using the four methods described above. Copper sulphate was further added to liver homogenates and served as a positive control.

### Example 5: Further Testing of AAV8.TTR.hATP7Bco Vector

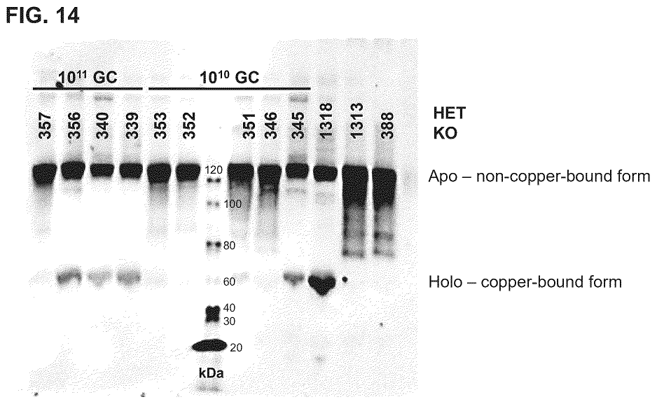

To determine the dose-dependent effects and minimal effective dose (MED) of the AAV8.EnTTR.TTR.hATP7Bco.PA75 vectors, both male and female 2 month-old ATP7B KO mice were injected intravenously with 1×10⁹, 1×10¹⁰ or 1×10¹¹ GC/mouse of the vector. The western blot for ceruloplasmin demonstrated an appearance of copper-bound ceruloplasmin in the serum of three out of four tested mice which received 1×10¹¹ GC of AAV8.EnTTR.TTR.hATP7Bco.PA75, suggesting an increase of copper extraction (FIG. 14). While upon treatment with 1×10¹⁰ GC of the same vector, one out of five tested mice showed copper-bound ceruloplasmin in the blood (FIG. 14). Those date validated that a single intravenous injection of AAV8.EnTTR.TTR.hATP7Bco.PA75at the dose of 1×10¹¹ GC per mouse successfully promoted the copper extraction in a murine model of Wilson's disease.

### Example 6: Truncated Vectors

Monogenic diseases are excellent candidates for gene replacement therapy approaches using AAV vectors. However, there is a limit to the size of the cDNA that can be packaged inside an AAV vector capsid. The wild type AAV genome is 4,700 bp, and requests to package larger genomes can reduce the integrity of the DNA sequence encapsulated within the AAV capsid (19). Previously, extensive research has been performed to investigate ways to reduce both the size of a given transgene and the transcriptional and polyA control sequences required for expression. One example of where this has been done successfully is development of a gene therapy vector for the treatment of hemophilia A, involving generation of B domain-deleted transgene sequence for human coagulation factor VIII that is 4,374 bp in length (19-21). For the treatment of Wilson's disease, the problem is increased as the ATP7B cDNA is 4,395 bp. Therefore, we chose to use a reduced size transthyretin enhancer and promoter (TTR) sequence for expression of a codon-optimized version of the human ATP7B transgene, in combination with a 75 bp synthetic poly A sequence (PA75) (19). The resulting AAV genome is 5.1 kb and was packaged within the AAV8 capsid.

Additionally, truncated hATP7Bco vectors, including AAV8.En34.TBG-S1.hATP7Bco (MBDIDel).PA75, AAV8.En34.TBG-S1.hATP7Bco (MBD2Del).PA75, AAV8.En34.TBG-S1.hATP7Bco (MBD3Del).PA75, AAV8.En34.TBG-S1.hATP7Bco (MBD1-2Del).PA75, AAV8.En34.TBG-S1.hATP7Bco (MBD1-4Del).PA75, AAV8.En34.TBG-S1.hATP7Bco (MBD1-5Del).PA75, AAV8.TBG.PI.hATP7Bco (MBD1-4Del).PA75, and AAV8.TBG.PI.hATP7Bco (MBD1-5Del).PA75, were designed, produced and injected into ATP7B KO mice intravenously at a dose of 3×10¹² GC/kg.

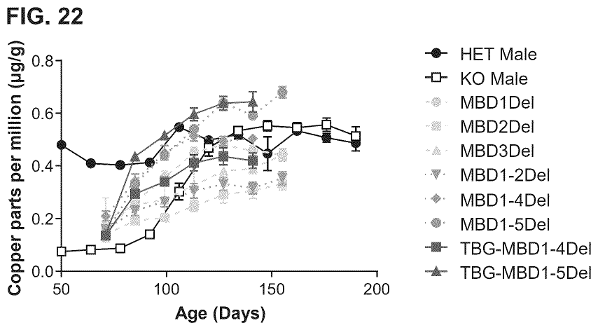

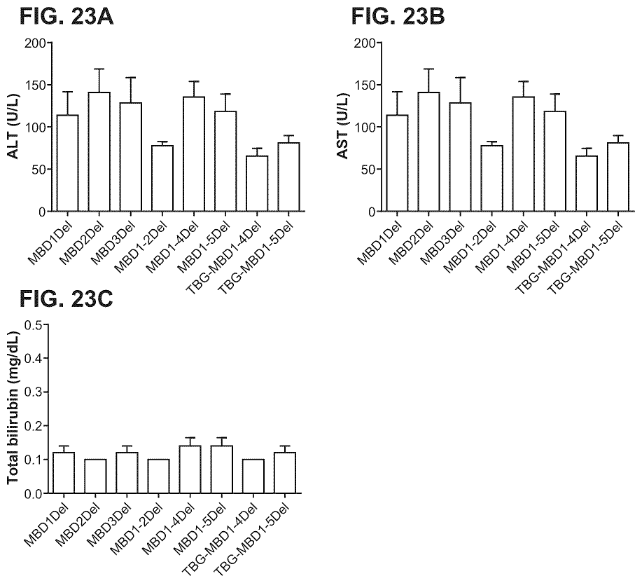

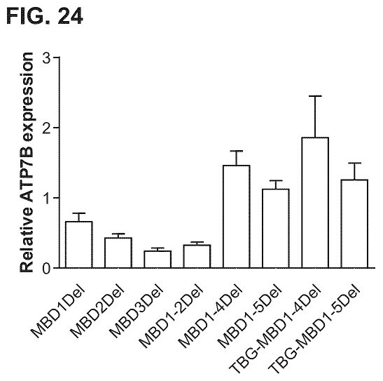

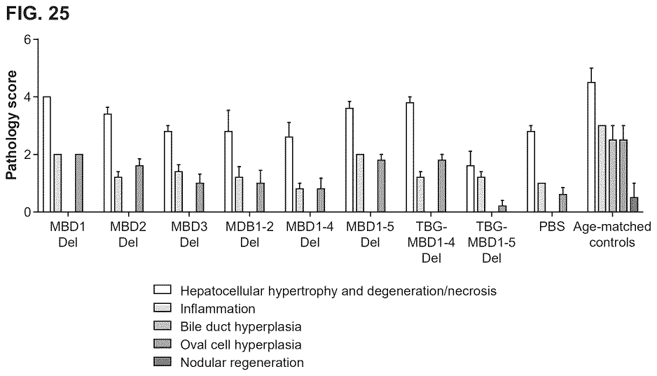

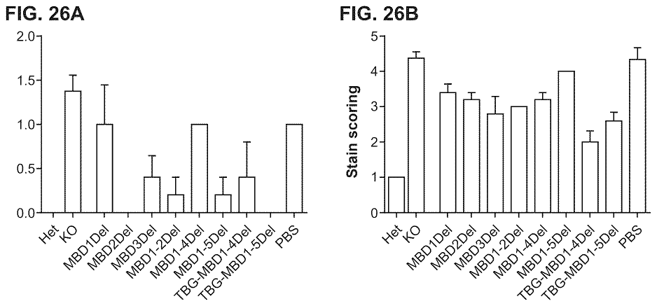

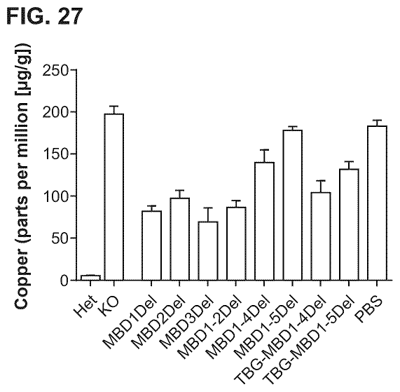

Atp7b KO mice treated with the various truncated vectors described above, and heterozygous littermates were studied and sacrificed at 6 months of age. Serum copper levels over time are shown in FIG. 22. ALT, AST and total bilirubin levels were obtained. FIG. 23. Relative ATP7B expression is shown in FIG. 24. H&E stain and then histopathology evaluated by pathologist against a scoring scheme, which is shown in FIG. 25. Fibrosis score and copper staining score by Timm's Stain are shown in FIG. 26. Liver copper levels at necropsy are shown in FIG. 27.

### Example 7: Materials and Methods for Above Experiments

### A. AAV Vector Production

All AAV vectors were produced by the Penn Vector Core at the University of Pennsylvania as described previously (10). Briefly, plasmids expressing a codon-optimized version of hATP7B (hATP7Bco) from a reduced sized transthyretin enhancer and promoter were packaged with the AAV8 viral capsid.

### B. Mice

Breeding pairs of heterozygous Atp7b^{+/-} mice were obtained from The Jackson Laboratory (Bar Harbor, ME, USA) and a colony was maintained at the University of Pennsylvania under specific pathogen-free conditions. All animal procedures and protocols were approved by the Institutional Animal Care and Use Committee (IACUC) of the University of Pennsylvania. Atp7b KO were generated and used for subsequent breeding. All pups generated from Atp7b KO mating pairs were fostered on Balb/c foster mothers within 72 hours of birth. Male and female Atp7b KO mice two months of age were injected IV with 10⁹-10¹¹ genome copies (GC)/mouse of AAV8.TTR.hATP7Bco via the tail vein (n=5/sex/group).

### C. Serum Analyses

Blood was collected at the indicated time points in serum separator tubes, allowed to clot, and serum was isolated by centrifugation at 3,500×g for 5 minutes at room temperature. Serum was analyzed for alanine aminotransferase (ALT), aspartate aminotransferase (AST), and total bilirubin levels by Antech Diagnostics (Irvine, CA, USA). Serum and urine was also analyzed for copper levels by Exova (Edinburgh, UK).

### D. Liver Copper Analysis

Liver samples taken at necropsy were analyzed for copper levels by Exova (Edinburgh, UK).

### E. Histopathology

Formalin-fixed paraffin-embedded tissue samples were sectioned and stained for hematoxylin and eosin (H&E) according to standard protocols. To detect liver fibrosis, Sirius Red staining was performed on paraffin sections. Sections were deparaffinized and stained for 90 minutes in a solution of 0.1% (w/v) Direct Red (Sigma), 4% (w/v) picric acid (Sigma), washed with 0.01 N HCl (2×1 min), dehydrated through an ethanol and xylene series, and cover slips applied.

### F. Timm's Copper Stain

Sections from formalin-fixed paraffin-embedded livers were dewaxed and incubated sequentially in 0.5% ammonium sulfide (5 min), deionized water (1 min rinse), 0.1N HCl (2-3 min), deionized water (2-3 min rinse), and developer (1 part 5% silver nitrate, 5 parts 2% (w/v) hydroquinone/5% (w/v) citric acid, for approximately 10 min). Liver sections from control animals (wild type and ATP7B KO) were included in each run and monitored for consistent staining intensity. Sections were finally washed in water, counterstained with Nuclear Fast Red, dehydrated, and cover slips applied.

### G. Histopathology Scoring

The histopathologic lesions were scored based on the following criteria. Hepatocellular hypertrophy and degeneration: 0, no significant lesions; 1, minimal karyocytomegaly (rare to occasional, 1-2 hepatocytes within a lobule), 2, mild karyocytomegaly (<10% of hepatocytes within a lobule); 3, moderate karyocytomegaly (10-50% of hepatocytes within a lobule) with hepatocyte dissociation and rare to few single cell necrosis; 4, severe karyocytomegaly (51-90% of hepatocytes within a lobule) with extensive hepatocyte dissociation and frequent single cell necrosis; relatively normal hepatic architecture is maintained; 5, marked karyocytomegaly (>90% of hepatocytes within a lobule) with lobular collapse and numerous single cell necrosis. Inflammation: 0, none; 1, mild-few aggregates within portal areas and rare foci within parenchyma; considered within normal limits; 2, moderate-extending into surrounding periportal hepatocytes or multifocally within parenchyma; 3, marked-bridging or dissecting hepatocytes or multifocal to coalescing within parenchyma. Bile duct hyperplasia: 0, none; 1, focal or multifocal within portal areas; 2, dissecting hepatocytes in periportal region; 3, bridging or dissecting hepatocytes with architectural distortion. Oval cell hyperplasia: 0, none; 1, focal or multifocal (periportal); 2, bridging or dissecting hepatocytes; 3, bridging or dissecting hepatoytes with architectural distortion. Nodular regeneration: 0, absent; 1, present.

The grading scheme for fibrosis based on Sirius red staining was derived from those reported in the literature (11): 0, none; 1, focal or multifocal; 2, bridging; 3, bridging with architectural disruption, with notation on whether the distribution was centrilobular, midzonal, periportal, or diffuse.

H. The grading scheme for copper accumulation based on Timm's staining was the same as has been previously described in the literature (12):1, absence or few copper-containing granules in the cytoplasm of an occasional hepatocyte; considered within normal limits; 2, obvious copper-containing granules in some centrilobular hepatocytes; considered within normal limits; 3, mild—numerous granules in most centrilobular hepatocytes (one-third of each lobule); 4, moderate—presence of numerous granules in all centrilobular and midzonal hepatocytes (approximately two-thirds of the hepatocytes in all lobules); 5 marked—abundant granules in more than two-thirds of the liver cells in all lobules.Statistical analysis

For all data, group average and standard error of the mean (SEM) was calculated and reported. Student's t test was performed to compare two groups and a one-way analysis of variance (AVOVA) with Tukey's multiple comparisons test was performed across groups and stratified by sex. The five pathology parameters were analyzed in vector administration and compared with those in age-matched uninjected Atp7b KO mice. Comparisons were carried out using the Wilcoxon rank sum test within the R program (version 3.3.1; cran.r-project.org). The difference in combined pathology parameters was also evaluated for each dose group compared to age-matched uninjected ATP7B KO mice using Fisher's combined probability test within the R program using function “sumlog” in the “metap” package. A p value of 0.05 was considered to be significant.

### Example 8: Discussion

Before a mouse model of disease can be used to assess the translatability of a therapeutic approach to the clinic, the model must be extensively characterized. While characterization of other mouse models of Wilson's disease have been performed (1, 15, 17), this study is the first detailed characterization of both the tx^{J} mouse strain and the evaluation of copper metabolism and liver disease following fostering of all Atp7b KO mice from birth. In the absence of the conflicting copper deficiency that occurs prior to weaning due to the Atp7b deficiency in the mammary glands of diseased mothers, the time line of disease progression in these mice can be more accurately determined by ensuring separation of the disease phenotype from any underlying issues related to copper deficiency prior to weaning. The development of liver disease and nodular regeneration in this mouse model was similar to what has been previously reported for the Atp7b^{-/-} strain developed by others (1, 17). However, the time line was slightly increased in this mouse strain, likely due to the fostering of pups from birth resulting in a continuous supply of copper.

In this Atp7b KO mouse model, the progression of disease was as follows: Severe copper accumulation in the liver was seen by Timm's copper stain at two months of age, but did decrease over time similar to that previously described by others (1, 15-17). Due to fostering of the pups, copper is likely accumulating in the liver of Atp7b KO mice from birth and reaching saturation levels around 2-3 months of age. Following development of liver disease at two months of age, copper is likely released into the serum, resulting in an apparent decrease in accumulation in hepatocytes and rising serum copper levels by 3-4 months of age. This rise in serum copper levels from initially low levels to those similar to heterozygous mice is different to what has been reported previously for another mouse model of Wilson's disease (17), where serum copper levels in the Atp7b^{-/-} strain are similar to that seen in wild type mice at 6 weeks of age and increase over time to 2 to 3-fold higher than wild type mice by 44 weeks of age.

We observed that the overflow of copper into urine starts at ˜3 months of age, which similarly could be due to hepatocellular necrosis and subsequent release of accumulated copper. Alternative mechanisms for excretion of copper via the kidney have been suggested, including lack of Atp7b activity in the kidney resulting in increased excretion (23-25), or accumulation of copper in the liver leading to downregulation of the liver copper transporter, Ctrl, and urinary excretion by a small copper carrier (26). Again, the time course of urinary copper excretion differs in this mouse model to that reported previously. Here, urinary copper excretion is initially similar in heterozygous and Atp7b KO mice, but increases in the KO mice over the course of the study. In comparison, urinary copper levels are 3-fold greater than wild type mice at 6 weeks of age in the Atp7b^{-/-} strain, increase up until 14-20 weeks old, and then decrease substantially at 20 weeks of age (26).

Hepatocellular hypertrophy, degeneration and necrosis peak at 6 months of age with likely concomitant observation of areas of hepatic nodular regeneration from this age onwards. This progression towards regeneration has been reported for other mouse models of Wilson's disease (17). However, unlike in the other mouse models, there was no evidence of cholangiocarcinoma in the Atp7b KO mouse described here. Regions of fibrosis were seen by 3 months of age, which rapidly increased in severity over time with architectural disruption by 7 months of age. While there are marked increases in serum transaminases by 3-4 months of age, increases in serum total bilirubin levels only start to occur at 9 months, indicating advanced liver disease.

Following characterization of this Atp7b KO mouse model, we developed a gene therapy approach for treatment of Wilson's disease. IV administration of an AAV8 vector expressing a codon-optimized version of the human ATP7B transgene into Atp7b KO mice at two months of age resulted in an increase in serum copper levels by two weeks post vector administration. The higher doses of vector evaluated here (>10⁹ GC/mouse) resulted in significant decreases in liver copper levels compared to age-matched, uninjected Atp7b KO mice. There was a significant, dose-dependent decrease in liver lesions, with only mild histopathological findings present in male mice injected with 10¹¹ GC/mouse and a complete lack of liver fibrosis. Therefore, administration of a gene therapy approach during the early stages of disease onset prevented liver damage and corrected copper metabolism in a mouse model of Wilson's disease.

All publications cited in this specification, as well as U.S. Provisional Patent Application Nos. 62/440,659 and 62/473,656, are incorporated herein by reference. Similarly, the SEQ ID NOs which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

Sequence Listing Free Text

| Seq ID NO | Free Text |
|---|---|
| 4-15 | <213> Artificial Sequence |
| | <223> constructed sequence |
| 17-35 | <213> Artificial Sequence |
| | <223> constructed sequence |

### REFERENCES

ADDIN EN.REFLIST 1. Buiakova O I, Xu J, Lutsenko S, Zeitlin S, Das K, Das S, Ross B M, et al. Null mutation of the murine ATP7B (Wilson disease) gene results in intracellular copper accumulation and late-onset hepatic nodular transformation. Hum Mol Genet 1999; 8:1665-1671.
2. Coronado V, Nanji M, Cox D W. The Jackson toxic milk mouse as a model for copper loading. Mamm Genome 2001; 12:793-795.
3. Theophilos M B, Cox D W, Mercer J F. The toxic milk mouse is a murine model of Wilson disease. Hum Mol Genet 1996; 5:1619-1624.
4. Sasaki N, Hayashizaki Y, Muramatsu M, Matsuda Y, Ando Y, Kuramoto T, Serikawa T, et al. The gene responsible for LEC hepatitis, located on rat chromosome 16, is the homolog to the human Wilson disease gene. Biochem Biophys Res Commun 1994; 202:512-518.
5. Wu J, Forbes J R, Chen H S, Cox D W. The LEC rat has a deletion in the copper transporting ATPase gene homologous to the Wilson disease gene. Nat Genet 1994; 7:541-545.
6. Terada K, Sugiyama T. The Long-Evans Cinnamon rat: an animal model for Wilson's disease. Pediatr Int 1999; 41:414-418.
7. Lutsenko S, Barnes N L, Bartee M Y, Dmitriev O Y. Function and regulation of human copper-transporting ATPases. Physiol Rev 2007; 87:1011-1046.
8. Michalczyk A, Bastow E, Greenough M, Camakaris J, Freestone D, Taylor P, Linder M, et al. ATP7B expression in human breast epithelial cells is mediated by lactational hormones. J Histochem Cytochem 2008; 56:389-399.
9. Bronson R T, Sweet H O, Davisson M T. Acute cerebral neuronal necrosis in copper deficient offspring of female mice with the toxic milk mutation. Mouse Genome 1995; 93:152-154.
10. Gao G, Lu Y, Calcedo R, Grant R L, Bell P, Wang L, Figueredo J, et al. Biology of AAV serotype vectors in liver-directed gene transfer to nonhuman primates. Mol Ther 2006; 13:77-87.
11. Bernard J M, Newkirk K M, McRee A E, Whittemore J C, Ramsay E C. Hepatic lesions in 90 captive nondomestic felids presented for autopsy. Vet Pathol 2015; 52:369-376.
12. Thornburg L P, Shaw D, Dolan M, Raisbeck M, Crawford S, Dennis G L, Olwin D B. Hereditary copper toxicosis in West Highland white terriers. Vet Pathol 1986; 23:148-154.
13. Stromeyer F W, Ishak K G. Histology of the liver in Wilson's disease: a study of 34 cases. Am J Clin Pathol 1980; 73:12-24.
14. Roberts E A, Schilsky M L, American Association for Study of Liver D. Diagnosis and treatment of Wilson disease: an update. Hepatology 2008; 47:2089-2111.
15. Biempica L, Rauch H, Quintana N, Sternlieb I. Morphologic and chemical studies on a murine mutation (toxic milk mice) resulting in hepatic copper toxicosis. Lab Invest 1988; 59:500-508.
16. Haywood S, Loughran M, Batt R M. Copper toxicosis and tolerance in the rat. III. Intracellular localization of copper in the liver and kidney. Exp Mol Pathol 1985; 43:209-219.
17. Huster D, Finegold M J, Morgan C T, Burkhead J L, Nixon R, Vanderwerf S M, Gilliam C T, et al. Consequences of copper accumulation in the livers of the Atp7b−/− (Wilson disease gene) knockout mice. Am J Pathol 2006; 168:423-434.
18. Smedley R, Mullaney T, Rumbeiha W. Copper-associated hepatitis in Labrador Retrievers. Vet Pathol 2009; 46:484-490.
19. Greig J A, Wang Q, Reicherter A L, Chen S J, Hanlon A L, Tipper C H, Clark K R, et al. Characterization of Adeno-Associated Viral Vector-Mediated Human Factor VIII Gene Therapy in Hemophilia A Mice. Hum Gene Ther 2017; 28:392-402.
20. Toole J J, Pittman D D, Orr E C, Murtha P, Wasley L C, Kaufman R J. A large region (approximately equal to 95 kDa) of human factor VIII is dispensable for in vitro procoagulant activity. Proc Natl Acad Sci USA 1986; 83:5939-5942.
21. Ward N J, Buckley S M, Waddington S N, Vandendriessche T, Chuah M K, Nathwani A C, McIntosh J, et al. Codon optimization of human factor VIII cDNAs leads to high-level expression. Blood 2011; 117:798-807.
22. Davidoff A M, Ng C Y, Zhou J, Spence Y, Nathwani A C. Sex significantly influences transduction of murine liver by recombinant adeno-associated viral vectors through an androgen-dependent pathway. Blood 2003; 102:480-488.
23. Bull P C, Thomas G R, Rommens J M, Forbes J R, Cox D W. The Wilson disease gene is a putative copper transporting P-type ATPase similar to the Menkes gene. Nat Genet 1993; 5:327-337.
24. Petrukhin K, Fischer S G, Pirastu M, Tanzi R E, Chernov I, Devoto M, Brzustowicz L M, et al. Mapping, cloning and genetic characterization of the region containing the Wilson disease gene. Nat Genet 1993; 5:338-343.
25. Tanzi R E, Petrukhin K, Chernov I, Pellequer J L, Wasco W, Ross B, Romano D M, et al. The Wilson disease gene is a copper transporting ATPase with homology to the Menkes disease gene. Nat Genet 1993; 5:344-350.
26. Gray L W, Peng F, Molloy S A, Pendyala V S, Muchenditsi A, Muzik O, Lee J, et al. Urinary copper elevation in a mouse model of Wilson's disease is a regulated process to specifically decrease the hepatic copper load. PLOS One 2012; 7:e38327.

1. A method of treating Wilson's Disease (WD) in a subject in need thereof, comprising administering to the subject a recombinant AAV (rAAV) comprising an adeno-associated (AAV) capsid and a vector genome packaged therein, said vector genome comprising:
(a) an AAV 5′ inverted terminal repeat (ITR) sequence;
(b) a transthyretin (TTR) promoter;
(c) a coding sequence encoding a truncated human copper-transporting ATPase 2 (ATP7B), wherein the truncated ATP7B comprises metal-binding domains (MBDs) 4-6 and lacks MBD 1-3; and
(d) an AAV 3′ ITR.

2. The method of claim 1, wherein the TTR promoter is a modified TTR promoter.

3. The method of claim 1, wherein the vector genome further comprises an enhancer.

4. The method of claim 3, wherein the enhancer is a TTR enhancer.

5. The method of claim 1, wherein the AAV capsid has tropism for liver.

6. The method of claim 5, wherein the AAV capsid is AAV8 capsid or an AAV9 capsid.

7. The method of claim 1, wherein the vector genome further comprises an intron.

8. The method of claim 7, wherein the intron is selected from a human beta-globin IVS2 intron or an SV40 intron.

9. The method of claim 1, wherein administering the rAAV comprises administering about 1×10¹² to about 1×10¹⁴ genome copies (GC) of the vector genome per kilogram body weight (GC/kg) in an aqueous suspension.

10. The method of claim 9, wherein about 5×10¹² to about 2×10¹³ GC/kg are administered to the subject.

11. The method of claim 9, wherein about 5×10¹² GC/kg are administered to the subject.

12. The method of claim 9, wherein about 1×10¹³ GC/kg are administered to the subject.

13. The method of claim 9, wherein about 2×10¹³ GC/kg are administered to the subject.

14. The method of claim 9, wherein the genome copy concentration is determined by quantitative PCR (qPCR) or digital droplet PCR (ddPCR).

15. The method of claim 9, wherein the aqueous suspension comprises the rAAV and at least one excipient selected from a surfactant, a preservative, or a buffer dissolved in an aqueous suspending liquid.

16. The method of claim 1, wherein the rAAV is administered intravenously.

17. A nucleic acid sequence encoding a truncated human copper-transporting ATPase 2 (ATP7B), wherein the truncated human ATP7B has an amino acid sequence corresponding to SEQ ID NO: 2, with a deletion of MBD1, MBD2, and MBD3.

18. The nucleic acid sequence of claim 17, wherein the deletion of MBD1 corresponds to the nucleic acid sequence of SEQ ID NO: 20, the deletion of MBD2 corresponds to the nucleic acid sequence of SEQ ID NO: 21, and the deletion of MBD3 corresponds to the nucleic acid sequence of SEQ ID NO: 22.

19. The nucleic acid sequence of claim 17, wherein the nucleic acid sequence encoding the truncated human ATP7B shares at least 95% sequence identity with the native human ATP7B coding sequence of SEQ ID NO: 3, excluding sequences encoding MBDs 1-3.


SEQUENCE LISTING (35 sequences)

SEQ ID NO: 1 - DNA, 4395 bp, synthetic construct
  misc_feature 1..4395: note = constructed sequence
  source 1..4395: mol_type = other DNA
atgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatc

SEQ ID NO: 2 - protein, 1465 aa, Homo sapiens
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIKSKVAPLSLGPIDIERLQSTNPKRPLSSANQNFNNSETLGHQGSHVVTLQLRIDGMHCKSCVLNIEENIGQLLGVQSIQVSLENKTAQVKYDPSCTSPVALQRAIEALPPGNFKVSLPDGAEGSGTDHRSSSSHSPGSPPRNQVQGTCSTTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 3 - DNA, 4395 bp, Homo sapiens
  source 1..4395: mol_type = other DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcaagagcaaagtggctcccttaagcctgggaccaattgatattgagcggttacaaagcactaacccaaagagacctttatcttctgctaaccagaattttaataattctgagaccttggggcaccaaggaagccatgtggtcaccctccaactgagaatagatggaatgcattgtaagtcttgcgtcttgaatattgaagaaaatattggccagctcctaggggttcaaagtattcaagtgtccttggagaacaaaactgcccaagtaaagtatgacccttcttgtaccagcccagtggctctgcagagggctatcgaggcacttccacctgggaattttaaagtttctcttcctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatc

SEQ ID NO: 4 - DNA, 34 bp, synthetic construct
  misc_feature 1..34: note = constructed sequence
  source 1..34: mol_type = other DNA
tgtttgctgcttgcaatgtttgcccattttaggg

SEQ ID NO: 5 - DNA, 100 bp, synthetic construct
  misc_feature 1..100: note = constructed sequence
  source 1..100: mol_type = other DNA
ctacctcgtgatcgcccggcccctgttcaaacatgtcctaatactctgtctctgcaagggtcatcagtagttttccatcttactcaacatcctcccagtg

SEQ ID NO: 6 - DNA, 42 bp, synthetic construct
  misc_feature 1..42: note = constructed sequence
  source 1..42: mol_type = other DNA
aggttaatttttaaactgtttgctctggttaataatctcagg

SEQ ID NO: 7 - DNA, 322 bp, synthetic construct
  misc_feature 1..322: note = constructed sequence
  source 1..322: mol_type = other DNA
aaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 8 - DNA, 178 bp, synthetic construct
  misc_feature 1..178: note = constructed sequence
  source 1..178: mol_type = other DNA
actcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagaca

SEQ ID NO: 9 - DNA, 477 bp, synthetic construct
  misc_feature 1..477: note = constructed sequence
  source 1..477: mol_type = other DNA
agggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 10 - DNA, 218 bp, synthetic construct
  misc_feature 1..218: note = constructed sequence
  source 1..218: mol_type = other DNA
tggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaata

SEQ ID NO: 11 - DNA, 190 bp, synthetic construct
  misc_feature 1..190: note = constructed sequence
  source 1..190: mol_type = other DNA
atttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 12 - DNA, 215 bp, synthetic construct
  misc_feature 1..215: note = constructed sequence
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 13 - DNA, 75 bp, synthetic construct
  misc_feature 1..75: note = constructed sequence
  source 1..75: mol_type = other DNA
aataaagtctgagtgggcggcagcctgtgtgtgcctgggttctctctgtcccggaatgtgcaaacaatggaggtg

SEQ ID NO: 14 - DNA, 168 bp, synthetic construct
  misc_feature 1..168: note = constructed sequence
  source 1..168: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacg

SEQ ID NO: 15 - DNA, 168 bp, synthetic construct
  misc_feature 1..168: note = constructed sequence
  source 1..168: mol_type = other DNA
cgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 17 - DNA, 3966 bp, synthetic construct
  misc_feature 1..3966: note = constructed sequence
  source 1..3966: mol_type = other DNA
atgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatc

SEQ ID NO: 18 - DNA, 3360 bp, synthetic construct
  misc_feature 1..3360: note = constructed sequence
  source 1..3360: mol_type = other DNA
atgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatc

SEQ ID NO: 19 - DNA, 2967 bp, synthetic construct
  misc_feature 1..2967: note = constructed sequence
  source 1..2967: mol_type = other DNA
atgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatc

SEQ ID NO: 20 - DNA, 4284 bp, synthetic construct
  misc_feature 1..4284: note = constructed sequence
  source 1..4284: mol_type = other DNA
atgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatc

SEQ ID NO: 21 - DNA, 4215 bp, synthetic construct
  misc_feature 1..4215: note = constructed sequence
  source 1..4215: mol_type = other DNA
atgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaaggaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatc

SEQ ID NO: 22 - DNA, 4317 bp, synthetic construct
  misc_feature 1..4317: note = constructed sequence
  source 1..4317: mol_type = other DNA
atgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatc

SEQ ID NO: 23 - DNA, 7944 bp, synthetic construct
  misc_feature 1..7944: note = constructed sequence
  source 1..7944: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctacctcgtgatcgcccggcccctgttcaaacatgtcctaatactctgtctctgcaagggtcatcagtagttttccatcttactcaacatcctcccagtggaattcatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggatggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaagtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaagtgaagtacgaccccagctgcacaagccccgtggccctgcagagagccattgaagctctgccccctggcaacttcaaagtgtccctgcctgatggcgccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgatggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaatgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctatgtgcaagcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaatgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaagtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaaatggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 24 - DNA, 7866 bp, synthetic construct
  misc_feature 1..7866: note = constructed sequence
  source 1..7866: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 25 - DNA, 7874 bp, synthetic construct
  misc_feature 1..7874: note = constructed sequence
  source 1..7874: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagaggttaatttttaaactgtttgctctggttaataatctcagggaattcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 26 - DNA, 7878 bp, synthetic construct
  misc_feature 1..7878: note = constructed sequence
  source 1..7878: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattcatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 27 - DNA, 7858 bp, synthetic construct
  misc_feature 1..7858: note = constructed sequence
  source 1..7858: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 28 - DNA, 7094 bp, synthetic construct
  misc_feature 1..7094: note = constructed sequence
  source 1..7094: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 29 - DNA, 7487 bp, synthetic construct
  misc_feature 1..7487: note = constructed sequence
  source 1..7487: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 30 - DNA, 7788 bp, synthetic construct
  misc_feature 1..7788: note = constructed sequence
  source 1..7788: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 31 - DNA, 7686 bp, synthetic construct
  misc_feature 1..7686: note = constructed sequence
  source 1..7686: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcatcctgggcatgacctgccagagctgcgtgaagtccatcgaggaccggatcagcaacctgaagggcatcatcagcatgaaggtgtccctggaacagggcagcgccaccgtgaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaaggaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 32 - DNA, 7755 bp, synthetic construct
  misc_feature 1..7755: note = constructed sequence
  source 1..7755: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcaaatacgtgccctctgtcgtgtgcctgcagcaggtgtgccaccagatcggcgacatgggcttcgaggcctctatcgccgagggaaaggccgcctcttggccctctagaagcctgcctgctcaggaagccgtcgtgaagctgcgggtggaagggatgacctgtcagtcctgcgtgtccagcatcgagggcaaagtgcggaagctgcagggcgtggtgcgcgtgaaagtgtctctgagcaaccaggaagctgtgatcacctaccagccctacctgatccagcccgaggacctgagggaccacgtgaacgatatgggatttgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 33 - DNA, 6438 bp, synthetic construct
  misc_feature 1..6438: note = constructed sequence
  source 1..6438: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 34 - DNA, 6831 bp, synthetic construct
  misc_feature 1..6831: note = constructed sequence
  source 1..6831: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 35 - DNA, 7437 bp, synthetic construct
  misc_feature 1..7437: note = constructed sequence
  source 1..7437: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacttaagtgtttgctgcttgcaatgtttgcccattttaggggaattcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagcggccgccaccatgcccgagcaggaaagacagatcaccgccagagagggcgccagccggaagatcctgagcaagctgagcctgcccaccagagcctgggagcccgccatgaagaagtccttcgccttcgacaacgtgggctacgagggcggcctggacggactgggaccatcttctcaggtggccacaagcaccgtgcgcgaggccgccatcaagagcaaggtggcccctctgtctctgggccccatcgacatcgagagactgcagagcaccaaccccaagcggcctctgagcagcgccaaccagaacttcaacaacagcgagacactgggccaccagggcagccacgtcgtgacactgcagctgcggatcgacggaatgcactgcaagagctgtgtgctgaacatcgaggaaaacatcggccagctgctgggagtgcagagcatccaggtgtcactggaaaacaagaccgcccaggtgaagtacgaccccagctgcacaagccccgtggccctgcagagagccattgaagctctgccccctggcaacttcaaagtgtccctgcctgacggagccgagggctccggaacagatcacagaagcagcagcagccacagccctggcagcccccctagaaatcaggtgcagggcacctgtagcaccaccctgatcgccattgccggcatgacatgcgccagctgcgtgcactctattgagggcatgatctcccagctggaaggcgtgcagcagatcagtgtgtctctggccgagggcaccgccacagtgctgtacaaccctagcgtgatcagccccgaagaactgagagccgccattgaggacatgggattcgaagccagcgtggtgtccgagagctgctccaccaaccctctgggcaatcacagcgccggcaacagcatggtgcagaccaccgacggcacccccaccagcgtgcaggaagtggccccacatacaggcagactgcccgccaatcacgcccccgatatcctggccaagagcccccagagtacaagagccgtggccccccagaagtgcttcctgcagatcaagggcatgacttgtgcctcttgtgtgtccaatatcgagcggaacctgcagaaagaggccggcgtgctgtctgtgctggtggctctgatggccggcaaggccgagatcaaatacgaccccgaagtgattcagcccctggaaatcgcccagtttatccaggacctgggctttgaagccgccgtgatggaagattacgccggctccgacggcaacatcgagctgaccatcaccggaatgacctgcgcctcctgtgtgcacaacattgagtccaagctgacccggaccaacggcatcacctacgcctctgtggctctggccacctccaaggccctcgtgaagttcgatcccgagatcatcggccccagggacatcatcaagatcatcgaagagatcggcttccacgccagcctggcccagaggaaccctaacgcccaccacctggaccacaagatggaaatcaagcagtggaagaaaagcttcctgtgcagcctggtgttcggcatccccgtgatggccctgatgatctacatgctgatccccagcaacgagccccaccagtccatggtgctggatcacaacatcatccccggcctgtctatcctgaacctgatcttcttcatcctgtgcaccttcgtgcagctgctgggcggctggtacttctacgtgcaggcctacaagtccctgcggcacagatccgccaacatggacgtgctgatcgtgctggccacatctatcgcctacgtgtactccctcgtgatcctggtggtggccgtggccgagaaagccgagagaagccctgtgaccttcttcgacaccccccctatgctgttcgtgtttatcgccctgggccggtggctggaacacctggccaaaagcaagaccagcgaggccctggctaagctgatgagtctgcaggccaccgaggccacagtcgtgaccctgggcgaggacaacctgatcatccgcgaggaacaggtgccaatggaactggtgcagcggggcgacatcgtgaaggtggtgcctggcggcaagttccccgtggacggaaaagtgctggaagggaataccatggccgacgagagcctgatcacaggcgaggccatgcccgtgaccaagaaacctggcagcacagtgatcgccggcagcatcaatgcccacggcagcgtgctgattaaggccacacacgtgggcaacgataccaccctggctcagattgtgaagctggtggaagaggcccagatgagcaaggcccccattcagcagctggctgaccggttcagcggctacttcgtgccctttatcatcatcatgagcaccctgacactggtcgtgtggatcgtgatcggctttatcgacttcggagtggtgcagagatacttccccaaccctaacaagcacatcagccagacagaagtgatcatcagattcgcctttcagaccagcatcaccgtgctgtgtatcgcctgcccctgtagcctgggactggccacacctaccgctgtgatggtgggaacaggcgtggccgctcagaacggcatcctgatcaaggggggcaagcctctggaaatggctcacaagatcaagaccgtgatgttcgacaagaccggcaccatcacccacggcgtgcccagagtgatgagagtgctgctgctgggggatgtggccaccctgcctctgagaaaggtgctggctgtcgtgggcacagccgaggctagctctgaacacccactgggagtggccgtgacaaagtactgcaaagaggaactgggcaccgaaaccctgggctactgcaccgactttcaggccgtgcctggctgtggcatcggctgcaaggtgtccaacgtggaaggcatcctggcccacagcgagaggccactgtctgcccctgccagccacctgaacgaggccggatctctgcccgccgaaaaggacgctgtgccccagaccttctctgtgctgattggcaacagagagtggctgcggcggaacggcctgaccatctcctccgatgtgtccgacgccatgaccgaccacgagatgaagggccagaccgccattctggtggccattgacggggtgctgtgcggcatgatcgcaatcgccgatgccgtgaaacaggaagcagcactggccgtgcacaccctgcagtctatgggagtggatgtggtgctgatcaccggcgacaacagaaagaccgccagggccattgccacccaggtgggcatcaacaaggtgttcgccgaggtgctgcccagccacaaagtggccaaggtgcaggaactgcagaacaaaggcaaaaaggtggccatggtgggagatggcgtgaacgactctcctgctctggcccaggcagatatgggcgtggccatcggcacaggcaccgacgtggcaattgaggctgctgacgtggtgctgattcggaacgacctgctggacgtggtggcctccatccacctgtccaagagaaccgtgcggcggatcagaatcaacctggtgctggcactgatctataacctcgtgggcatccctatcgccgctggcgtgttcatgcctatcggaatcgtgctgcagccctggatgggctctgccgccatggctgcaagctccgtgtctgtggtgctgtccagcctgcagctgaagtgctacaagaagcccgacctggaaagatacgaggcccaggcccacggacacatgaagcctctgacagcctcccaggtgtccgtgcacatcggcatggacgacagatggcgggacagccctagagccaccccttgggatcaggtgtcatacgtgtcacaggtgtccctgagcagcctgaccagcgacaagcccagcagacatagcgccgctgccgacgacgatggggacaagtggtccctgctgctgaacggccgggatgaggaacagtacatctgataagcatgcaataaagtctgagtgggcggcagcctgtgtgtgcctgggttctctctgtcccggaatgtgcaaacaatggaggtgctcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg